SEQUENCE LISTING (2 sequences)

SEQ ID NO: 1 - protein, 12 aa, artificial sequence
  peptide substrate for Abl kinase enzyme
EAIYAAPFAKKK

SEQ ID NO: 2 - protein, 15 aa, artificial sequence
  peptide substrate for Scl kinase enzyme
KVEKIGEGTYGVVYK

(19) United States
(12) Patent Application Publication
(10) Pub. No.: US 2010/0226853 A1
(43) Pub. Date: Sep. 9, 2010

(54) Fluorine-18 derivatives of dasatinib and uses thereof

(76) Inventors: Darren R. Veach, New Rochelle, NY (US); Nagavara Kishore Pillarsetty, Jackson Heights, NY (US); Steven M. Larson, New York, NY (US); Elmer B. Santos, Bronx, NY (US); Mohammad Namavari, Plao Alto, CA (US)

Correspondence Address: Benjamin Aaron Adler;ADLER & ASSOCIATES, 8011 Candle Lane, Houston, TX, 77071, US

(21) Appl. No.: 12/798,462

(22) Filed: Apr. 5, 2010

Related U.S. Application Data
(63) Continuation-in-part of application No. PCT/US2008/011509, filed on Oct. 6, 2008.
(60) Provisional application No. 60/997,783, filed on Oct. 4, 2007.

(51) Int. Cl. A61K 51/04; C07D 417/14; A61K 51/12

(52) U.S. Cl. 424/1.21; 544/295; 424/1.89; 424/1.29

(57) ABSTRACT

Provided herein are [¹⁸F]-labeled compounds having a chemical structure:

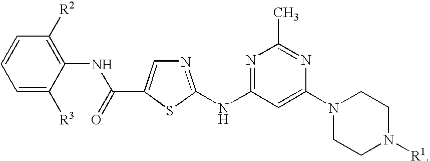

R¹ is ¹⁸F, 1-piperazinyl-4-CH₂CH₂—¹⁸F or 1-piperazinyl-4-CH₂CH₂OCH₂CH₂—¹⁸F, R² is CH₃ or ¹⁸F and R³ is Cl or ¹⁸F, such that only one of R¹, R² and R³ comprise an ¹⁸F. Also provided are methods for in vivo imaging using the [¹⁸F]-labeled compounds, particularly methods of imaging utilizing positron emission tomography. These methods are effective for diagnosing a pathophysiological condition susceptible to treatment with kinase inhibitor(s) in a subject, or for determining whether a cancer in a subject that is susceptible to being treated with a kinase inhibitor has developed resistance or increased sensitivity to the same and for maximizing tumor response to akinase inhibitor with minimal toxicity to the subject.

### CROSS-REFERENCE TO RELATED APPLICATIONS

This is a continuation-in-part application which claims benefit of priority under 35 U.S.C. §120 of international application PCT/US2008/011509, filed Oct. 6, 2008, which claims benefit of priority under 35 U.S.C. §119(e) of provisional application U.S. Ser. No. 60/997,783, filed Oct. 4, 2007, now abandoned, the entirety of both of which are hereby incorporated by reference.

### BACKGROUND OF THE INVENTION

1. Field of the Invention

The present invention relates to kinases and positron emission tomography (PET) visualization of certain pathophysiological conditions in vivo. More specifically, the present invention relates to a fluorine-18 derivatives and analogs of Dasatinib and their uses in PET to visualize pathophysiological conditions associated with a kinase activity in vivo.

2. Description of the Related Art

A focus of modern medicine is to develop care that is individualized to each patient. An important facet of this has been kinase inhibitor therapy, and signal transduction modulation in general. Another key aspect of customized care is obtaining a detailed disease profile through non-invasive medical imaging techniques such as PET and using this to assess disease status and determine the optimal course of treatment. Molecular and functional imaging with radiotracers such as [¹⁸F]-fluoro-2-deoxy-D-glucose (FDG) and [¹⁸F]-3′-deoxy-3′-fluorothymidine (FLT) are particularly useful as surrogate markers in cancer diagnosis and management (1-2). However, more sophisticated tumoral information is necessary to predict response or observe the onset of drug resistance. Radiolabeled small molecule imaging modalities that are matched to a given kinase inhibitor and are capable of querying a specific molecular target are one possible solution.

PET is a non-invasive nuclear medicine imaging technique that produces a virtual three-dimensional computer image that, visualizes, quantifies and localizes areas of radioactivity of a given energy within the tissues and organs of a living subject. PET uses positron-emitting radioisotopes with short half lives (HL) such as fluorine-18 (¹⁸F), ¹¹C (HL:˜20 min), ¹³N (HL:˜10 min), ¹⁵O (HL:˜2 min), and ¹⁸F (HL:˜110 min). The type of biochemical activity, such as enzyme function, or biological target, such as a receptor, that is imaged by PET depends upon the type of radioactive tracer used.

A radiotracer is a molecule chemically-conjugated to an amount of radioactive isotope far below therapeutic concentrations and concentrations that saturate binding sites and that participates in specific biochemical processes or binds to specific biological target(s) of interest. A radiotracer is typically administered to a subject intravenously. As the radiotracer circulates throughout the body, it distributes according to the specifically-related biochemical activity, or concentration of the biological target within individual tissues and organs. The PET scanner localizes and quantifies this activity within the body of the subject by detecting the source of photons emitted in the decay of the tracer-radioisotope. Computer analysis of this data generates PET images, which are interpreted by physicians.

Normal Abl and Src kinases are expressed in a variety of tissues and are tightly regulated and inactive most of the time. Both have many functions and associations in vivo, but generally, Src regulates cell adhesion and motility, while Abl is involved in cytoskeletal reorganization (3) and cell death signaling (4). In some leukemias, a reciprocal t(9;22) translocation between the ABL and BCR genes forms the Philadelphia chromosome (Ph), whose mutant gene product, Bcr-Abl, is a constitutively activated tyrosine kinase. Bcr-Abl causes chronic myelogenous leukemia (CML) and some types of acute lymphoblastic leukemia (ALL) (5). Src tyrosine kinase is activated and/or overexpressed in numerous malignancies, mutated in a few examples and is often associated with increased motility, invasiveness or metastasis in cancer (6). The abundance, activation and disregulation of Bcr-Abl and Src in cancer make these kinases attractive targets for drug development and molecular imaging.

Imatinib, a Bcr-Abl tyrosine kinase inhibitor, is one of the most well known molecularly targeted therapeutics and has revolutionized treatment of CML (7-8). Imatinib is also approved for gastrointestinal stromal tumor (GIST) therapy and acts via inhibition of c-Kit receptor tyrosine kinase (9). While imatinib has been a major breakthrough, resistance to kinase inhibitor therapy arises from a number of mechanisms including kinase-domain point mutations (pre-existing or acquired), upregulation of Bcr-Abl, activation of alternate, compensatory kinase pathways (Src family), and drug transporters (10). The issue of resistance is complicated further by residual quiescent cancer stem cells that are less susceptible to therapy possibly by one or more of the aforementioned mechanisms (11-12). These issues have fueled the development of a number of next-generation Bcr-Abl inhibitors (13-14). Dasatinib (BMS-354825) is a high affinity dual Src/Abl and c-Kit inhibitor recently approved for all categories of imatinib-refractory CML and Ph+ ALL (15-16). Dasatinib is effective in many imatinib resistant Bcr-Abl kinase domain mutants, but the “gatekeeper” mutants like T315I or F317L remain problematic (16).

Dasatinib is an anticancer drug. Treatments with dasatinib employ either a fixed dosage (70 mg twice-daily) or the conventional ‘maximum tolerated dose’ approach, wherein drug dosage starts low and is increased until the patient experiences toxicity. Administered orally, the absorption and pharmacokinetics of dasatinib, i.e., the amount of ingested dasatinib that could actually reach the tumor, varies among individuals, influenced by gastric pH & food content, drug interactions, and other factors. A standard starting dose is 70 mg twice-daily, though no linear dose-response relationship is evident, at levels both above and below 70 mg twice-daily. Yet dasatinib-toxicity is clearly dose-related. Severe myelosuppression occurs in >50% of patients, with diarrhea and severe hemorrhage (including CNS) as other major toxicities.

In vitro, dasatinib-sensitive solid tumor cell lines demonstrate a conventional dose-response curve (48). Hence, in cancer patients treated with oral doses of dasatinib, it would be valuable for clinicians to know what amount of a given dose actually reaches tumors, in vivo; possessing this knowledge should help clinicians predict tumor response, allowing earlier modification of therapeutic regimens, when satisfactory response is unlikely. Detecting changes in tumor pharmacokinetics may also provide a novel means of identifying the onset of chemoresistance to Dasatinib.

¹⁴C- and ³H-labeled (beta-emitting) radiotracers are produced routinely in drug development, but kinase inhibitors bearing positron-emitting isotopes are much less developed. No kinase inhibitor-based imaging probe exists yet for routine use in humans. Thus far, the majority of effort has been by vanBrocklin, Mishani and others on quinazoline-type small-molecule probes for EGFR tyrosine kinase, which is overexpressed in some cancers (17-22). More recently, Wang, et al. reported the synthesis of [¹¹C]-gefitinib (23) and [¹⁸F]-sunitinib (24).

Recently, [¹⁸F]-FLT PET was used to distinguish bone marrow in patients with myeloproliferative disorders from normal (27) [¹¹C]-AG957 was the first example of a Bcr-Abl-targeted radiotracer specifically developed for PET, but this tracer suffers from inherent chemical instability and weak target binding relative to newer inhibitors (28-29). An [¹²⁴I]-pyridopyrimidinone derivative (30) which binds tightly to Bcr-Abl, among other kinases, has been reported but does not possess an ideal logP. LogP is the ratio of concentrations of a compound in the two phases of a mixture of two immiscible solvents at equilibrium, and is a measure of differential solubility of the compound between these two solvents. Recently, Fowler reported [¹¹C]-imatinib imaging in baboon (31). Generally, ¹⁸F is more convenient for PET imaging studies, as it has a 110-minute half-life, unlike ¹¹C (T_{1/2}=20 min).

Thus, there is an increasing need in the art for radiofluorinated tracers for PET imaging. Specifically, the prior art is deficient in a better PET tracer for the Abl and c-kit and other kinases in the form of a ¹⁸F derivative of Dasatinib that has both favorable physical properties and strong target binding. The present invention fulfills the longstanding need in the art.

### SUMMARY OF THE INVENTION

The present invention is directed to a [¹⁸F]-labeled compound for in vivo imaging of cells or tissue using positron emission tomography having a chemical structure:

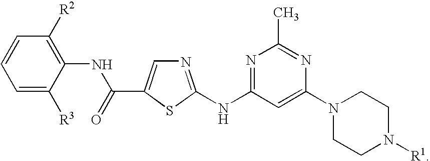

In the compound R¹ is ¹⁸F, 1-piperazinyl-4-CH₂CH₂—¹⁸F or 1-piperazinyl-4-CH₂CH₂OCH₂CH₂—¹⁸F, R² is CH₃ or ¹⁸F and R³ is Cl or ¹⁸F, such that only one of R¹, R² and R³ comprise an ¹⁸F. The present invention is directed to a related compound further comprising a physiologically acceptable a physiologically acceptable solubilizing agent or a delivery vehicle incorporating the compound.

The present invention also is directed to a related [¹⁸F]-labeled compound having the chemical structure

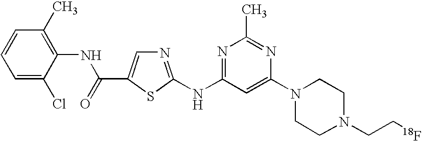

and to compositions and solubilized formulations comprising an excipient, such as a solubilizing agent, or to a nanoparticle or liposomal formulation.

The present invention also is directed to a method for diagnosing a pathophysiological condition susceptible to treatment with a kinase inhibitor in a subject in need of such diagnosis. The method comprises administering a sufficient amount of the [¹⁸F]-labeled compound as described herein to the subject to provide an imageable concentration therewithin whereupon the subject is imaged using positron emission tomography (PET). A determination that the intensity of the label in any body area of the subject is increased in comparison with normal background indicates that the individual has a condition that is susceptible to being treated with the kinase inhibitor. The present invention is directed to a related method further comprising treating the pathophysiological condition with a pharmacologically effective dose of one or more of the kinase inhibitors. The present invention is directed to another related method further comprising synergistically treating the cancer by administering one or both of a chemotherapeutic agent or a radiotherapeutic agent.

The present invention is directed to another related method for monitoring the susceptibility of the pathophysiological condition to treatment with the kinase inhibitor to determine whether resistance or increased sensitivity to the treatment has developed. The method comprises administering another imageable amount of the compound to the subject and imaging the subject using PET. The intensity of the label in a body area associated with the athophysiological condition is compared to an immediately previously obtained label-intensity, where a decrease in intensity compared to the previous intensity indicates that the pathophysiological condition is more resistant to treatment with the kinase inhibitor(s) or, alternatively, where an increase in intensity compared to the previous intensity indicates that the pathophysiological condition is more sensitive to treatment. The steps may be repeated for continuous monitoring.

The present invention is directed further still to an in vivo method using positron emission tomography for imaging cells or tissue having a kinase activity associated with a pathophysiological condition in a subject. The method comprises administering to the subject a sufficient amount of the [¹⁸F]-labeled compound provided herein to provide an imageable concentration of the compound in the cells or tissue. Emissions from the [¹⁸F] label comprising the compound are detected thereby forming an image of the cells or tissue.

The present invention is directed further still to a method for maximizing tumor response to a kinase inhibitor with minimal toxicity therefrom in a subject having a cancer. The method comprises administering to the subject an imageable amount of the [¹⁸F]-labeled compound described herein and imaging the subject a first time using positron emission tomography (PET). A dose of the kinase inhibitor is administered to the subject, an imageable amount of the [¹⁸F]-labeled compound is administered to the subject and the subject is imaged a second time using positron emission tomography (PET). The imaged tumor uptake of the [¹⁸F]-label in the second PET scan is compared with the imaged tumor uptake of the [¹⁸F]-label in the first PET scan, where a disappearance of [¹⁸F]-label intensity for any one tumor in the subject in the second scan compared to the intensity in the first scan indicates that the tumor is being treated at a sufficient therapeutic concentration of kinase inhibitor. If the intensity of the label remains the same or decreases, but does not disappear, the tumor requires an increase in the therapeutic dose of the kinase inhibitor, thereby maximizing tumor response. The present invention is directed to a related method further comprising designing a therapeutic regimen to treat the cancer with minimal toxicity to the subject based on the saturation dose of the kinase inhibitor.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. The above may be better understood by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIGS. 1A-1B are synthetic schema showing the synthesis of an unlabeled (¹⁹F) fluorinated derivative of Dasatinib (FIG. 1A) and two radiosynthetic routes to an [¹⁸F] derivative of Dasatinib (FIG. 1B).

FIGS. 2A-2B are cavity-depth (FIG. 2A) and Connolly (FIG. 2B) surface renderings of 5 docked into Abl kinase domain.

FIGS. 3A-3F illustrate inhibition of cellular proliferation of M07e/p210^{bcr-abl} (R10 neg) (FIG. 3A), M07e (FIG. 3B), and K562 (FIG. 3C) cell lines with fluorinated analog 5 versus Dasatinib (FIGS. 3D-3F).

FIG. 4 depicts a HPLC chromatogram showing coelution of [¹⁸F]-5 with co-injected non-radioactive reference ¹⁹F compound 5. Phenominex Luna C₁₈ 4.6×250 mm, 5μ, isocratic 60% NaOAc/40% CH₃CN, 1.0 mL/min. (Δ=0.4 min between detectors).

FIG. 5 illustrates inhibitory activity of 5 on 21 kinases at 10 nM.

FIG. 6 illustrates microPET imaging of a K562 xenograft in a mouse with [¹⁸F]-5 from 60-75 min.

FIGS. 7A-7D are [¹⁸F]-5 microPET images of a SCID mouse bearing H1975 lung cancer xenograft on its right shoulder (FIG. 7A) and H1975-DR lung cancer xenograft on its left shoulder (FIG. 7B). FIGS. 7A-7B are transaxial images showing bilateral tumor uptake (FIG. 7A) and competitive inhibition of tracer uptake (FIG. 7B) by unlabeled Dasatinib. FIGS. 7C-7D are coronal images showing bilateral tumor uptake (FIG. 7C) and competitive inhibition of tracer uptake in tumor and organs (FIG. 7D).

FIGS. 8A-8B are a comparison between biodistribution (FIG. 8A) and tumor to organ ratio (FIG. 8B) of saline and captisol formulated [¹⁸F]-5 in mice bearing CWR22Rv1 xenografts (n=5, Avg. 191 μCi).

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device, compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the term “dasatinib” refers to the chemical compound N-(2-chloro-6-methylphenyl)-2-[[6-[4-(2-hydroxyethyl)-1-piperazinyl]-2-methyl-4-pyrimidinyl]amino]-5-thiazole carboxamide monohydrate.

As used herein, the term “subject” is any recipient of compound [¹⁸F]-5 or other [¹⁸F] labeled dasatinib derivative or analog.

In one embodiment of the present invention there is provided an [¹⁸F]-labeled compound for in vivo imaging of cells or tissue using positron emission tomography having a chemical structure:

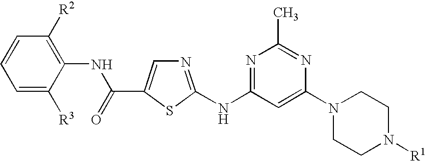

where R¹ is ¹⁸F, 1-piperazinyl-4-CH₂CH₂—¹⁸F or 1-piperazinyl-4-CH₂CH₂OCH₂CH₂—¹⁸F; R² is CH₃ or ¹⁸F; and R³ is Cl or ¹⁸F, such that only one of R¹, R² and R³ comprise an ¹⁸F.

Further to this embodiment the compound may be solubilized with a physiologically acceptable solubilizing agent. Preferably, the solubilizing agent may be a sulfobutyl beta-cyclodextrin, sulfated-beta-cyclodextrin or 2-hydroxypropyl-beta-cyclodextrin. Alternatively, the [¹⁸F]-labeled compound may be incorporated into a nanoparticle or liposome. In one aspect of these embodiments R² is CH₃ and R³ may be Cl. In this aspect R¹ is 1-piperazinyl-4-CH₂CH₂—¹⁸F, R² is CH₃ and R³ is Cl.

In a related embodiment the present invention provides a [¹⁸F] labeled compound having the chemical structure:

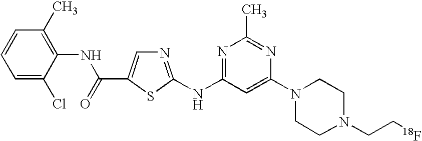

Further to this embodiment the composition may comprise the [¹⁸F] labeled compound and a physiologically acceptable excipient or delivery vehicle incorporating the compound. For example, the excipient may be a sulfobutyl beta-cyclodextrin, sulfated-beta-cyclodextrin or 2-hydroxypropyl-beta-cyclodextrin. Alternatively, the delivery vehicle may be a nanoparticle or liposome.

In another related embodiment the present invention provides a solubilized radiotracer formulation comprising the [¹⁸F] labeled compound of claim 16 and a physiologically acceptable solubilizing agent. The solubilizing agent may be as described supra.

In another embodiment of the present invention there is provided a method for diagnosing a pathophysiological condition susceptible to treatment with a kinase inhibitor in a subject in need of such diagnosis, comprising the steps of administering a sufficient amount of the [¹⁸F]-labeled-compound described supra to the subject to provide an imageable concentration therewithin; imaging the subject using positron emission tomography; and determining whether the intensity of the label in any body area of the subject is increased in comparison with normal background, wherein an increase in intensity of the labeling indicates that the individual has a condition that is susceptible to being treated with the kinase inhibitor.

Further to this embodiment, the method may comprise treating the pathophysiological condition with a pharmacologically effective dose of one or more of the kinase inhibitors. Further still the method may comprise synergistically treating the cancer by administering one or both of a chemotherapeutic agent or a radiotherapeutic agent.

Yet further still to these embodiments, the method may comprise monitoring the susceptibility of the pathophysiological condition to treatment with the kinase inhibitor to determine whether resistance or increased sensitivity to the treatment has developed. In this further embodiment, the step of monitoring susceptibility may comprise administering another imageable amount of the [¹⁸F]-labeled compound as described supra to the subject; imaging the subject using PET; and comparing the intensity of the label in a body area associated with the pathophysiological condition to an immediately previously obtained label-intensity, where a decrease in intensity compared to the previous intensity indicates that the pathophysiological condition is more resistant to treatment with the kinase inhibitor(s) or where an increase in intensity compared to the previous intensity indicates that the pathophysiological condition is more sensitive to treatment. Yet further still the method may comprise repeating the steps to continue monitoring changes in resistance or sensitivity to treatment.

In all embodiments the kinase inhibitor may be dasatinib. Also, the pathophysiological condition may be a cancer or may have an inflammatory component. In addition the pathophysiological condition is associated with a disregulated or an up-regulated kinase signaling pathway.

In yet another embodiment of the present invention there is provided an in vivo method for imaging cells or tissue having a kinase activity associated with a pathophysiological condition in a subject, comprising the steps of administering to the subject a sufficient amount of a [¹⁸F]-labeled compound as described supra to provide an imageable concentration of the derivative or analog in the cells or tissue; and detecting emissions from the [¹⁸F] label comprising the derivative or analog, thereby forming an image of the cells or tissue.

In this embodiment the cells or tissue may comprise a tumor. Also, the pathophysiological condition and the kinase, particularly a tyrosine kinase, may be as described supra.

In yet another embodiment of the present invention there is provided a method for maximizing tumor response to a kinase inhibitor with minimal toxicity therefrom in a subject having a cancer, comprising the steps administering to the subject an imageable amount of the [¹⁸F]-labeled compound described supra; imaging the subject a first time using positron emission tomography (PET); administering to the subject a dose of the kinase inhibitor; administering to the subject an imageable amount of the [¹⁸F]-labeled compound; imaging the subject a second time using positron emission tomography (PET); comparing the imaged tumor uptake of the [¹⁸F]-label in the second PET scan with the imaged tumor uptake of the [¹⁸F]-label label in the first PET scan, where a disappearance of [¹⁸F]-label intensity for any one tumor in the subject in the second scan compared to the intensity in the first scan indicates that the tumor is being treated at a sufficient therapeutic concentration of the kinase inhibitor and where if the intensity of the label remains the same or decreases, but does not disappear, the tumor requires an increase in the therapeutic dose of the kinase inhibitor, thereby maximizing tumor response.

Further to this embodiment the method comprises designing a therapeutic regimen to treat the cancer with minimal toxicity to the subject based on the saturation dose of the kinase inhibitor. In both embodiments the kinase inhibitor may be a tyrosine kinase as described supra.

Provided herein are the radiosynthesis, biological evaluation and in vivo micro-PET imaging of a fluorine-18 radiotracer. The [¹⁸F]-labeled compounds may be based on a potent, multi-targeted kinase inhibitor, for example, but not limited to, dasatinib, which is approved for the treatment of imatinib-resistant CML and Ph+ ALL. The [¹⁸F]-labeled radiotracer compounds may be derivatives or analogs of dasatinib and may have the chemical structure:

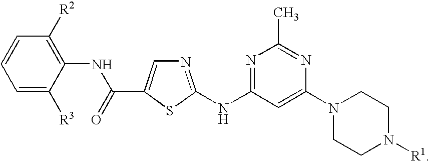

The R¹ substituent may be ¹⁸F, 1-piperazinyl-4-CH₂CH₂—¹⁸F or 1-piperazinyl-4-CH₂CH₂OCH₂CH₂—¹⁸F. The R² substituent may be CH₃ or ¹⁸F and the R³ substituent may be Cl or ¹⁸F. The [¹⁸F]-label may comprise one substituent in a radiotracer compound. Preferably, R¹ is ¹⁸F, 1-piperazinyl-4-CH₂CH₂—¹⁸F or 1-piperazinyl-4-CH₂CH₂OCH₂CH₂—¹⁸F and more preferably, R¹ is 1-piperazinyl-4-CH₂CH₂—¹⁸F. Thus, the more preferred [¹⁸F]-labeled dasatinib may have the chemical structure:

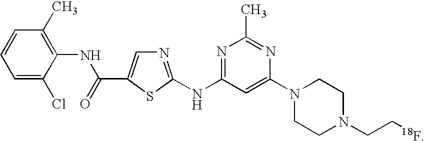

When considering the structure of dasatinib, the hydroxyethylpiperazinyl moiety was ideal for derivatization based on binding orientation. Chemically, the most straightforward approach was at the same site; N-alkylation of the unsubstituted piperazine with a simple fluorine-containing group or activated precursor for fluoride displacement (32). Thus, an analog of dasatinib bearing an [¹⁸F] fluoroethyl substituent was constructed. N-(2-Fluoroethyl)piperazines are not widely reported, but a successful synthesis of activated ethylpiperazines and subsequent displacement with ¹⁸F has been described (33). Sterically, hydroxyl-to-fluoro or hydroxy-to-chloro-to-fluoro substitution is tolerated well, so long as the hydroxyl is not involved in critical H-bonding. Particularly, N-(2-Chloro-6-methylphenyl)-2-(6-(4-(2-fluoroethyl)piperazin-1-yl)-2-methylpyrimidin-4-ylamino)thiazole-5-carboxamide (Compound 5) has similar target selectivity to dasatinib in vitro and retains strong anti-tumor potency. Generally, compound [¹⁸F]-5 and all precursors and intermediates are synthesized using known and standard chemical synthetic techniques, as described (33, 49).

In addition, the present invention provides compositions comprising the [¹⁸F]-labeled Dasatinib derivatives or analogs and a physiologically acceptable carrier or excipient. For example, the hydrophobic [¹⁸F]-labeled radiotracer compounds may be delivered in saline or solubilized with an appropriate solubilizing agent such as sulfobutyl beta-cyclodextrin, sulfated beta-cyclodextrin or 2-hydroxypropyl-beta-cyclodextrin. For example, the sulfobutyl beta-cyclodextrin Captisol® effectively solubilizes these radiotracer compounds such that about 99-100% of the radiolabel is available for imaging upon delivery. Alternatively, the compounds of the present invention may be complexed with or incorporated into a nanoparticle or liposome, as is known or standard in the art. As such, the present invention also provides a solubilized radiotracer formulation comprising the [¹⁸F] radiolabeled compounds solubilized in a suitable agent or comprising a nanoparticle or liposome.

The present invention provides imaging methods using the [¹⁸F]-labeled Dasatinib derivative or analog. These [¹⁸F]-labeled dasatinib derivatives or analogs may be administered in amounts sufficient to produce an imageable concentration in cells or tissues particularly associated with a pathophysiological condition, such as, but not limited to a cancer, e.g., a leukemia, or having an inflammatory component, as are known in the art. These [¹⁸F]-labeled compounds are particularly suited to imaging via positron emission tomography. One of ordinary skill in the art is well-suited to determine amounts of the [¹⁸F]-labeled compounds to administer to a subject, the route of administration and the PET imaging conditions necessary to obtain a useable image.

Generally, as the [¹⁸F]-labeled compounds are effective to bind to or competitively inhibit a kinase, particularly a tyrosine kinase, it is contemplated that the [¹⁸F]-labeled compounds provided herein are suitable to image and to locate within a body mass a kinase associated with a pathophysiological condition. For example, the pathophysiological condition may be associated with a disregulated or an up-regulated kinase signaling pathway. Examples of imageable tyrosine kinases are Abl, Ack, Csk, EphA2, EphB4, Kit, PDGFR-alpha, Src or Tec.

Particularly, as a probe, the [¹⁸F]-labeled compound [¹⁸F]-5 had significant K562 tumor uptake in mice, and thus can be used as a molecularly-targeted PET imaging probe with in vivo models of systemic CML, GIST and other malignancies involving Abl, Src and Kit. Thus, [¹⁸F]-5 is effective to visualize tumor characteristics on a molecular level, non-invasively, such as the existence or emergence of drug-resistant leukemia in bone marrow among others. Established proliferative imaging modalities like [¹⁸F]-FLT or [¹⁸F]-FDG are valuable, but cannot give the same information about the molecular changes occurring during disease progression or the emergence of resistance.

Thus, the present invention provides imaging methods useful as diagnostic tools or indicators. Upon diagnosing a pathophysiological condition, for example, but not limited to, a cancer, treatment regimens may be designed utilizing Dasatinib or another kinase inhibitor. One of ordinary skill in the art is well suited to design such a regimen, including determining dosages and times and route of administration depending on the subjects age, sex, health, and progression or remission of the disease.

One mechanism of tumor resistance to dasatinib therapy involves changes in the tumor receptor-targets that prevent dasatinib-binding. It is an object of the present invention to provide an assay that can detect the inability of dasatinib to bind to its tumor target-receptors which predicts tumor resistance to dasatinib therapy or alternatively, to detect an increase in chemosensitivity. This spares patients needless toxicity and allows clinicians to make earlier changes in therapeutic regimens. It is contemplated that therapeutic regimens may include synergistic treatment with a chemotherapeutic agent or radiotherapeutic agent. Such synergistic treatment is effective to increase chemo- and/or radio-sensitivity to the treatment. For example the Abl signaling pathway is known to be involved in radioresistance/radiosensitivity and when inhibited can confer increased radiosensitivity to a cell line.

Another mechanism of tumor resistance to dasatinib therapy involves increases in the tumor receptor-targets, requiring increased doses of the therapeutic drug. It is an object of the present invention to provide an assay that can detect the inability of dasatinib to completely inhibit its tumor target-receptors which predicts tumor resistance to dasatinib therapy. This allows clinicians to make earlier changes in therapeutic regimens.

The lack of a clear linear dose-response relationship, for oral dasatinib therapy, suggests that tumor & systemic pharmacokinetics vary widely, among patients; correlating tumor concentrations of dasatinib or other kinase inhibitor (by PET) to tumor response should allow clinicians a clearer understanding of the dose-response relationship for individual cancer patients. Additionally, [¹⁸F]-dasatinib PET allows this tumor dose-response relationship to be studied on a tumor-by-tumor basis within the same patient, in metastatic disease, which no other assay can do. Multiple biopsies of scattered metastases has never been standard clinical practice; visualizing heterogeneous tumor pharmacokinetics, in metastases, may clarify the meaning of changes in tumor size, post-therapy.

[¹⁸F]-dasatinib imaging provides for correlation or comparison of tumor response to tumor dosage. Determining how therapeutic dose levels of dasatinib or other kinase inhibitor affect the tumor accumulation of [¹⁸F]-labeled dasatinib, compared to a pre-treatment PET scan, can be effective to determine changes in tumor [¹⁸F]-dasatinib uptake which then serves as an index of the amount of tumor target therapeutic drug saturation. Determining whether the specific tumor or metastatic tumor is saturated or not by the specific therapeutic dasatinib or other kinase inhibitor levels administered to a patient in need of such treatment, using [¹⁸F]-dasatinib and PET scan, can be an indicator of whether the dasatinib or other kinase inhibitor dose given the patient should be increased, decreased or unmodified. The ability to visualize the saturation of dasatinib or other kinase inhibitor binding sites, in a tumor, in vivo, by PET, allows the relationship between the ingested dose levels of oral dasatinib or other kinase inhibitor therapy and clinical efficacy, as specific tumor shrinkage or lack thereof can be visualized, to be determined.

Thus, the present invention also provides an assay which can detect tumor saturation, by dasatinib or other kinase inhibitor, which is useful as a tool for maximizing tumor therapy-response while minimizing drug toxicity. Prescribing doses in excess of the dosage at which tumor saturation occurs increases the risk of chemotoxicity without increasing tumor therapy-response. Yet prescribing a kinase inhibitor dose which fails to saturate tumor target-receptors yields a suboptimal tumor therapy-response. Therefore, an important object of the present invention is that PET imaging with [¹⁸F]-dasatinib changes the dosage goal in the treatment of a cancer patient from maximum tolerated dose to maximum tumor dose or saturation point.

The following example(s) are given for the purpose of illustrating various embodiments of the invention and are not meant to limit the present invention in any fashion.

### Example 1

### Methods and Materials

### Chemicals and MS/NMR

All chemicals and solvents were obtained from Sigma-Aldrich (Milwaukee, Wis.) or Fisher Scientific (Pittsburgh, Pa.) and used without further purification. ¹H, ¹³C, and ¹⁹F NMR spectra were recorded on a Bruker AMX-400 at 400, 100 and 376 MHz, respectively or a Bruker AVANCE II 500 at 500, 125 or 470 MHz, respectively. Chemical shifts (δ) are determined relative to CDCl₃ (referenced to 7.27 ppm (δ) for ¹H-NMR and 77.0 ppm for ¹³C-NMR) or DMSO-d₆ (referenced to 2.49 ppm (δ) for ¹H-NMR and 39.5 ppm for ¹³C-NMR). The internal reference for ¹⁹F-NMR was CFCl₃ (0.0 ppm (δ)). Coupling constants (J) are given in Hertz and spectral splitting patterns are designated as singlet (s), doublet (d), triplet (t), quadruplet (q), multiplet or overlapped (m), and broad (br).

Low resolution mass spectra (ionspray, a variation of electrospray) were acquired on a Perkin-Elmer Sciex API 100 spectrometer. HRMS service was obtained from the Mass Spectrometry Lab at UIUC and acquired on a Micromass 70-SE-4F spectrometer using FAB⁺ ionization. HPLC was performed on a Jasco (Easton, Md.) system comprised of a PU-2089plus pump, UV-2075plus UVNIS detector, LCNetII/ADC data acquisition system and Windows PC running EZChrom Elite v3.1.4 software. Flash chromatography was performed using Merck silica gel 60 (mesh size 230-400 ASTM) or using an Isco (Lincoln, Nebr.) CombiFlash Companion or SQ16x flash chromatography system with RediSep columns (normal phase silica gel (mesh size 230-400 ASTM) and Fisher Optima™ grade solvents. Microwave reactions were performed in a CEM Discover microwave reaction system (Matthews, N.C.). Thin-layer chromatography (TLC) was performed on E. Merck (Darmstadt, Germany) silica gel F-254 aluminum-backed plates with visualization under UV (254 nm) and by staining with potassium permanganate or ceric ammonium molybdate. Molecular modeling was performed using SYBYL 7.1 (Tripos Inc., St. Louis, Mo.) on an Intel Xeon PC workstation running RedHat Enterprise Linux 3.

### Radiosynthesis

All HPLC solvents were filtered (0.45 μm, nylon, Alltech) prior to use. Water (ultra-pure, ion-free) was obtained from a Millipore Alpha-Q Ultra-pure water system. Sep-Pak® cartridges were obtained from Waters Corporation (Milford, Mass.). Radio-TLC was performed on silica gel plates (5×20 cm; 250 μm thickness; Aldrich, Milwaukee, Wis.) and analyzed with a BioScan AR-2000 Imaging Scanner (BioScan Inc., Washington D.C.) HPLC was performed using a Shimadzu (Columbia, Md.) system composed of a C-18 reversed-phase column (Phenominex Luna analytical 4.6×250 mm or semi-prep 10×250 mm, 5μ, 1.0 or 4.0 mL/min, 50 mM pH 5.5 NaOAc/CH₃CN), two LC-10AT pumps, an SPD-M10AVP photodiode array detector and a BioScan Flow Count radiodetector using a 25_{—}25 mm NaI(TI) crystal. Radioactivity was assayed using a Capintec CRC-15R dose calibrator (Ramsey, N.J.).

No-carrier-added [¹⁸F] fluoride ion was produced by the ¹⁸O(p,n)¹⁸F nuclear reaction by bombardment of an enriched [¹⁸O] H₂O target with 11 MeV protons using an EBCO-TR19 cyclotron. The ¹⁸F fluoride ion was trapped on an Accell™ Plus QMA ion-exchange cartridge (Waters).

### Computational Chemistry

Molecular modeling and graphics renderings were performed using the SYBYL 7.1 (Tripos Associates Inc., St. Louis, Mo.) software package on an Intel Xeon PC workstation running RedHat Enterprise Linux 3. The Abl kinase:PD166326 kinase inhibitor cocrystal structure was used as the initial model. This file can be obtained from the protein databank (coordinate file 10PK, www.rcsb.org).

A more appropriate starting structure would be the Abl:Dasatinib cocrystal structure reported by Tokarski, et al. (43), but at the time this work was initiated, the coordinate file 2GQG had not been released on the RCSB. The atom types for the inhibitor were corrected, hydrogen atoms were added to the protein and the C and N endgroups were fixed using the SYBYL/BIOPOLYMER module. Protein and inhibitor atomic charges were calculated using MMFF94 force field. The complex was minimized using the SYBYL gradient convergence method with an MMFF94s force field and 0.05 kcal/mol·Å rms gradient as the convergence criterion. All heavy atoms (inherent to the crystal structure) were constrained in an aggregate during minimization.

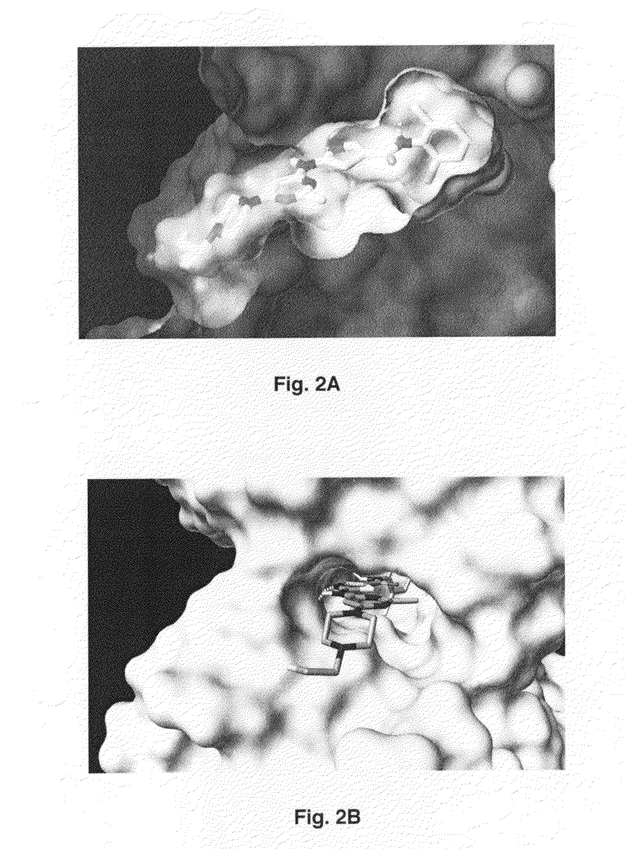

To create the Abl kinase:Compound 5 model, the inhibitor in the AblK:PD166326 cocrystal structure was replaced with compound 5 in an orientation that preserves the H-bond donor acceptor pair at Met318 and directs the fluoroethylpiperazinyl moiety out into solvent-exposed area (FIGS. 2A-2B). The inhibitor atoms were allowed to move freely for minimization. Conformational analysis run on the ligand showed that the fluoroethyl sidechain has considerable freedom of motion. Several lowest energy conformers of the terminal fluoroethyl group were found and minimized, but ultimately showed negligible differences in energy.

### Octanol/Water Partition Coefficient Determination

Octanol/water partition coefficients were determined for each radiotracer by shaking 370 KBq (10 μCi) of each radioligand with 10 mL of n-octanol and 10 mL of water for 2 hours. Octanol and deionized water were presaturated for at least 24 hours prior to use. The two layers were separated and spun in a centrifuge at 1000 g for 20 minutes. 1 mL samples were recovered with a syringe with a 25 gauge needle from each solvent and counted in a gamma counter. The samples of both layers were also analyzed for impurities by HPLC and the partition coefficient determined.

### Protein Binding Assay

Protein binding of the radiotracers were determined adding 37 kBq (1 μCi) of each radioligand to samples of 1% bovine serum albumin and 1 mL of fresh human serum. The protein was precipitated by adding 1 mL of ice cold 20% trichloroacetic acid and the suspension centrifuged and washing with 1 mL of 20% ice cold trichloroacetic acid. The protein pellets and supernatants were counted in a gamma counter to determine the protein binding of the radioligands.

### Tyrosine Kinase Activity Assays

Abl and Src kinase activity was measured according to Trentham (44) with some modifications. For Abl, the reaction was in 25 mM Hepes buffer pH 7.5, 10 mM MgCl₂, 2 mM DTT, 20 mM β-glycerol phosphate, 0.1 mM Na₃VO₄, 120 μM β-NADH, 500 μM phosphoenolpyruvate, and including 3.1 μg/ml L-lactic dehydrogenase, 6.67 μg/ml pyruvate kinase, 0.005% Tween 80, 1% DMSO, 5 nM Abl kinase (Invitrogen), 30 μM peptide substrate EAIYAAPFAKKK (SEQ ID NO: 1) (˜1×Kₘ), and 200 mM ATP (˜10×Kₘ). For Src, the same reaction conditions were used except for the following: 10 mM MnCl₂ (instead of MgCl₂), 20 nM Src kinase, 300 μM KVEKIGEGTYGVVYK-OH peptide, SEQ ID NO: 2 (˜1×Km), 200 μM ATP (˜3×Kₘ). Kinase was preincubated with inhibitor at 37° C. for 10 min prior to starting the reaction upon addition of ATP; Assay was carried out in 384 well clear plates (Corning) and absorbance was measured on SpectraMax plate reader (Molecular Devices). Reaction rates were plotted against inhibitor concentration and fitted using SigmaPlot 9.0 (Systat Software Inc.).

In addition to the rigorous IC₅₀ determinations in Abl and Src kinase above, a set of 21 tyrosine kinases were evaluated using Carna Biosciences' (Kobe, Japan) QuickScout™ service to measure kinase activity inhibition by compound 5 at 10 nM. Staurosporine was used as a control/benchmark inhibitor. Literature K_{d} values for Dasatinib are included for comparison. An ELISA-based assay was used in which the phosphorylation of an oligopeptide substrate was detected by an HRP-conjugated anti-p-Tyr (PY20) probe. The concentration of ATP was at the approximate Kₘ of each kinase (0.5-100 μM). The data appears in Table 2 in Example 3.

### Tumor Cell Culture

The immortalized human hematopoietic Philadelphia chromosome-positive cytokine independent R10(−) M07e^{p210} cell line (46) was maintained in Iscove's modified Dulbecco's medium (Life Technologies, Inc., Grand Island, N.Y.) containing 10% FCS (Hyclone, Logan, Utah). The parental M07e megakaryoblastic cell line was a kind gift of Brian Druker and was maintained in the presence of 50 ng/mL kit ligand (SCF) as described (46-47) K562 was obtained from the ATCC. K562 was maintained in suspension in 90% RPMI 1640 medium supplemented with 10% FBS, 2 mM L-glutamine, 10 mM HEPES, 1 mM sodium pyruvate, 1.5 g/L sodium bicarbonate, 4.5 g/L L-glucose, 10% FBS, 100 IU/mL penicillin and 100 μg/ml streptomycin. Tumor cell cultures were maintained in a humidified atmosphere with 5% CO₂ at 37° C. (NuAire).

### Cellular Proliferation Assay

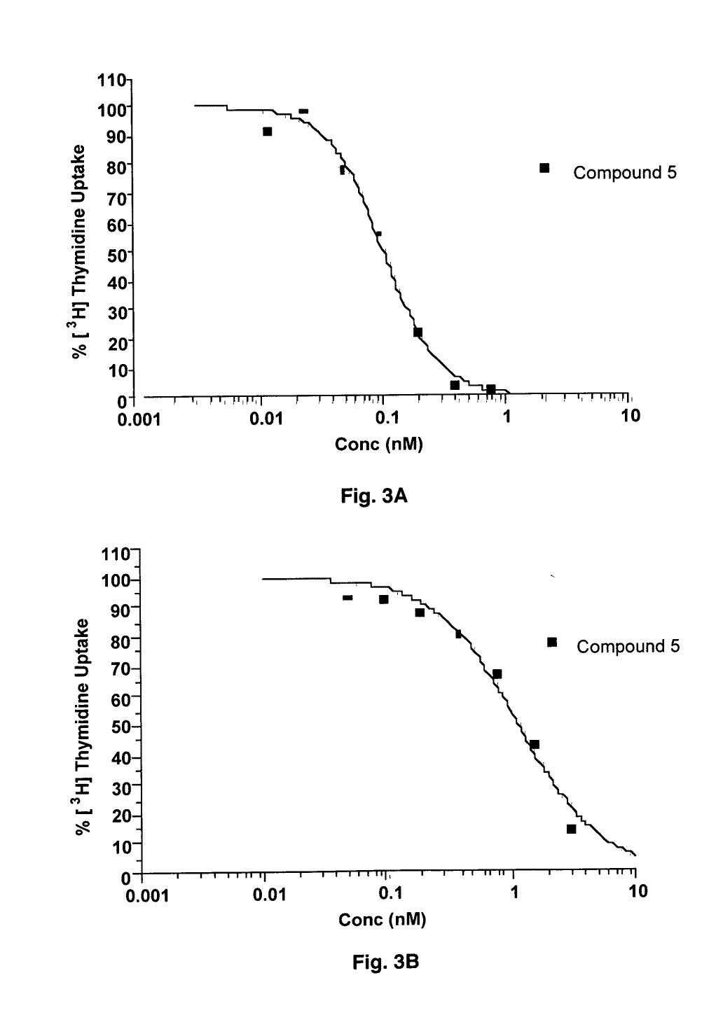

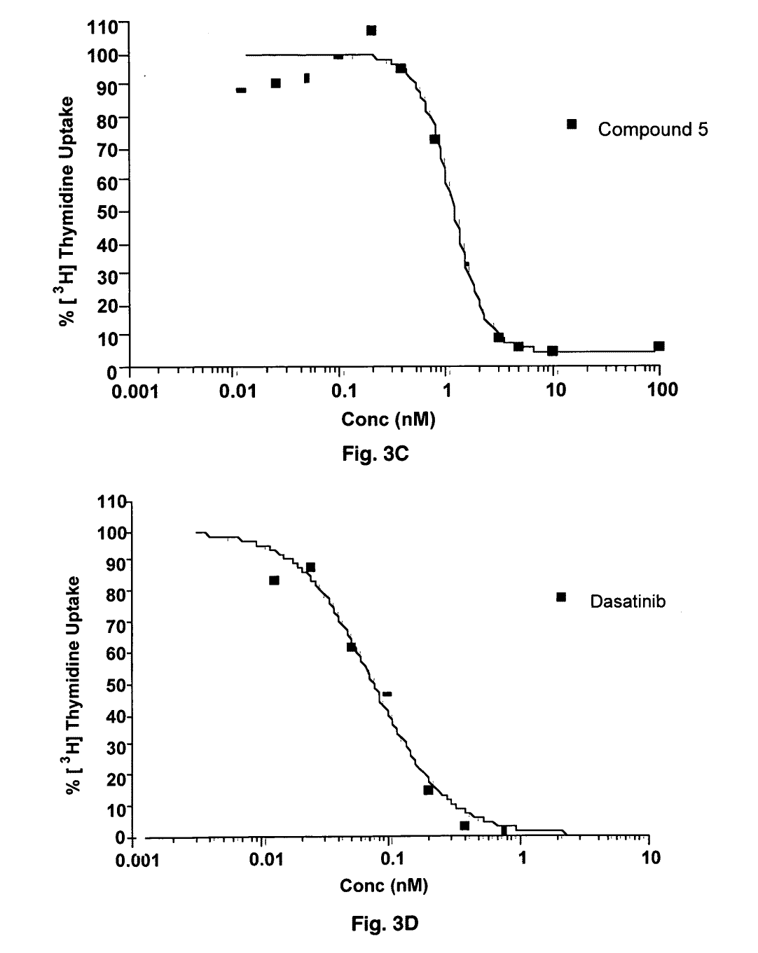

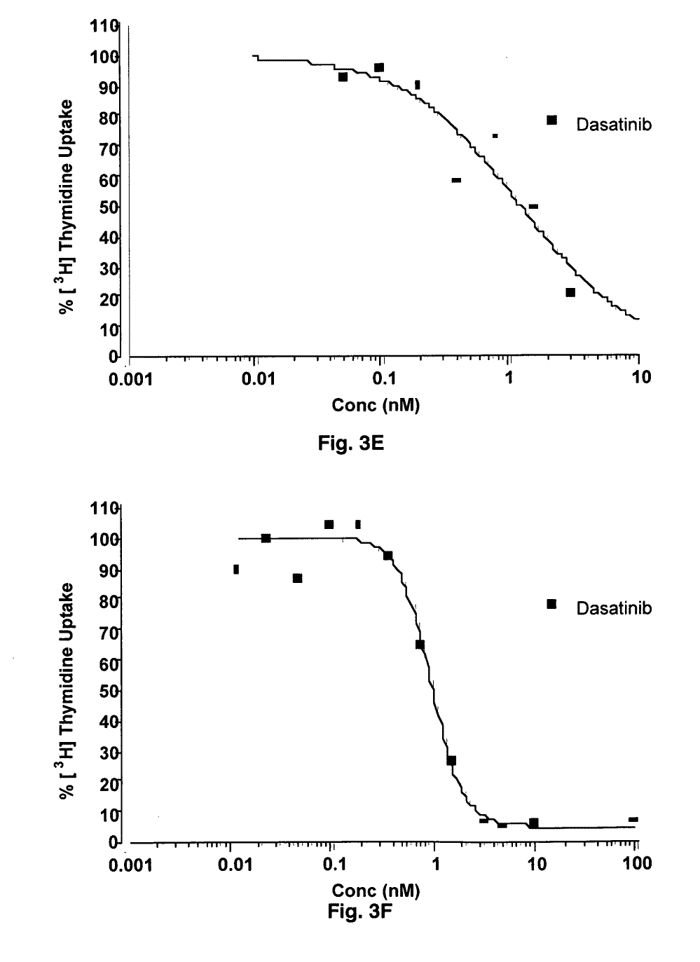

FIGS. 3A-3F shows cell growth determined by a [³H]thymidine uptake assay. Cells (10⁴ cells/well) were cultured in 96-well, round-bottomed plates (Fisher Scientific) with diluted DMSO (control) or with varying concentrations of Dasatinib or fluorinated derivative 5 that were resuspended in DMSO for 48 h at 37° C. [³H]Thymidine was added at a concentration of 1 μCi/well, and cells were incubated for an additional 18 h. Cells were harvested with the Unifilter system, scintillation fluid (25 μl/well) was added to each well, and [³H]thymidine incorporation was determined on a Packard Scintillation Counter. Data points for all assays were obtained in triplicate, and background incorporation from cell-free wells was determined and subtracted from all data points. The data was analyzed using a non-linear regression fit to a sigmoidal dose-response curve using Prism 4 (GraphPad Software, Inc).

### Toxicity in Mice

Mice were injected i.v. with unlabeled (¹⁹F) reference compound 5 in the tail vein and sacrificed using carbon dioxide at 30, 60 and 120 min post injection. Each group contained three mice. A total of 24 eight-week-old B6D2F1 mice (average initial weight was 22.9 g for male mice and 18.5 g for female mice) were used in the acute toxicity study. There were five males and five females in either control or treatment group. The treatment group received one dose of compound 5 (0.1 mg/kg) intravenously through tail vein injection and the control group received the same amount of vehicle (85% beta-hydroxypropyl cyclodextrine, 5% DMSO, 10% EtOH). All animals were observed for 14 days following treatment. Observation showed no apparent anemia, no weight loss, no agitation, no tachypnea, no GI disturbances and no apparent neurological dysfunction in all mice. In both the control and treatment groups, male mice gained on average 1.8 g in body weight and females gained on average 0.5 g. Mice were sent for pathology, including complete blood cell count, complete chemistry panel and complete necropsy. Two intact male mice and two intact female mice were also sent and used as references. Results showed no significant abnormalities in any control or treated mouse compared with intact mice. There was no evidence of drug-induced or vehicle induced organ toxicities. In conclusion, fluoro derivative 5, administered at 0.1 mg/Kg intravenously, is safe and nontoxic in B6D2F1 mice.

### In Vivo Metabolism in Mice

Mice were injected i.v. with [¹⁸F]-5 in the tail vein and sacrificed using carbon dioxide at 30, 60 and 120 min post injection. Each group contained three mice. Immediately after sacrifice, about 0.5 ml of blood was collected by cardiac puncture and deposited in a 1.5 ml Eppendorf tube. Disodium EDTA (2.5 mg) was used as anticoagulant. The samples were then maintained at 4° C. for subsequent procedures. The total radioactivity in each total blood sample was counted. The samples were then centrifuged at 4° C. at 2200×g. Radioactivity of the serum and pellet measured and about 50% of the total radioactivity was retained in the pellet. The serum was transferred to a 1.5 ml Eppendorf tube containing about 700 ml of 60% acetonitrile in water and centrifuged again to precipitate any residual proteins. The supernatant was analyzed using HPLC and examined for metabolites. HPLC was carried out on a C-18 Shimadzu 4.6×250 mm HPLC column and eluted under gradient conditions 80% A (pH 5.5 50 mM NaOAc):20% B (CH₃CN) to 20% A:80% B at 1 ml/min. Radioactivity was detected using Packard Radiomatic FLO-One/beta detector equipped with a PET flow cell containing BGO (bismuthgermanate) windows.

Animal Imaging with PET

All animal studies were carried out within the framework of an institutional IACUC approved protocol (No. 86-02-020). Athymic nu/nu mice (National Cancer Institute, Bethesda, Md.) were inoculated subcutaneously onto the right shoulder with 1×10⁷ K562 cells mixed with Matrigel (BD Biosciences, San Jose, Calif.). K562 is a chronic myelogenous leukemia cell line cultured with IMDM (Iscove's modified Dulbecco's medium; prepared in-house) containing 4 mM L-glutamine, 1.5 g/L sodium bicarbonate, and 10% fetal bovine serum.

Three weeks following tumor inoculation, PET images of mice were obtained using FOCUS 120 microPET™ scanner (Siemens Preclinical Solutions, Knoxyille, Tenn.) with [¹⁸F]-5. Mice were injected intravenously (via tail vein) with 14±1 MBq (375±25 μCi) of [¹⁸F]-5 and imaged 60 minutes later under 2% (at 1 L/min) isoflurane anesthesia (Forane, Baxter Healthcare, Deerfield, Ill.). Image acquisition time was 15 min (t=60 to 75 min) using a 250-750 keV energy window and a 6 nsec timing window. List-mode data were sorted into sinograms by Fourier re-binning and reconstructed by filter back-projection without attenuation correction. Count data in the reconstructed images were converted to activity concentration (i.e. % of the injected dose per gram (% ID/gm)) using a system calibration factor determined using a ¹⁸F-filled mouse-sized phantom. Visualization and analyses of microPET images were carried out using AsiPRO™ software (Siemens Preclinical Solutions, Knoxyille, Tenn.).

### Example 2

### Chemical Synthetic Scheme 1

### N-(2-Chloro-6-methylphenyl)-2-(6-(4-(2-chloroethyl)piperazin-1-yl)-2-methylpyrimidin-4-ylamino)thiazole-5-carboxamide, 2

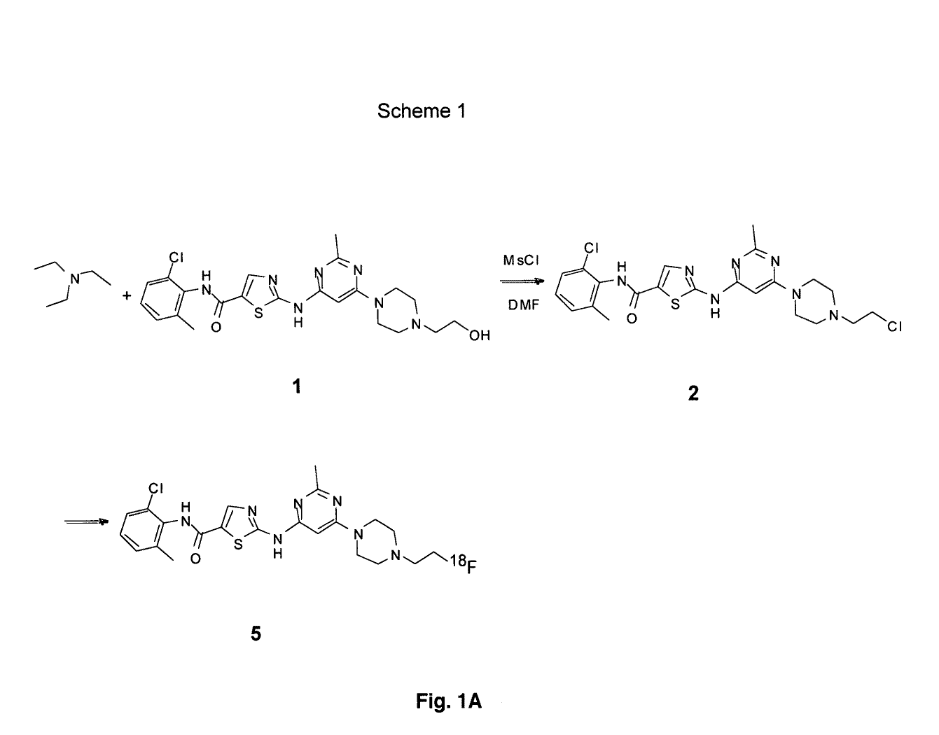

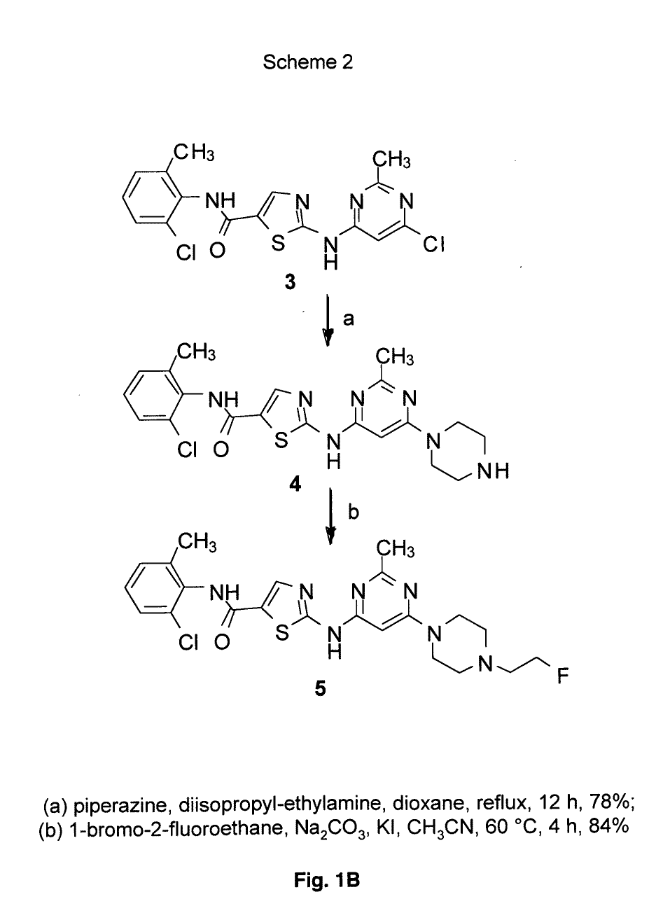

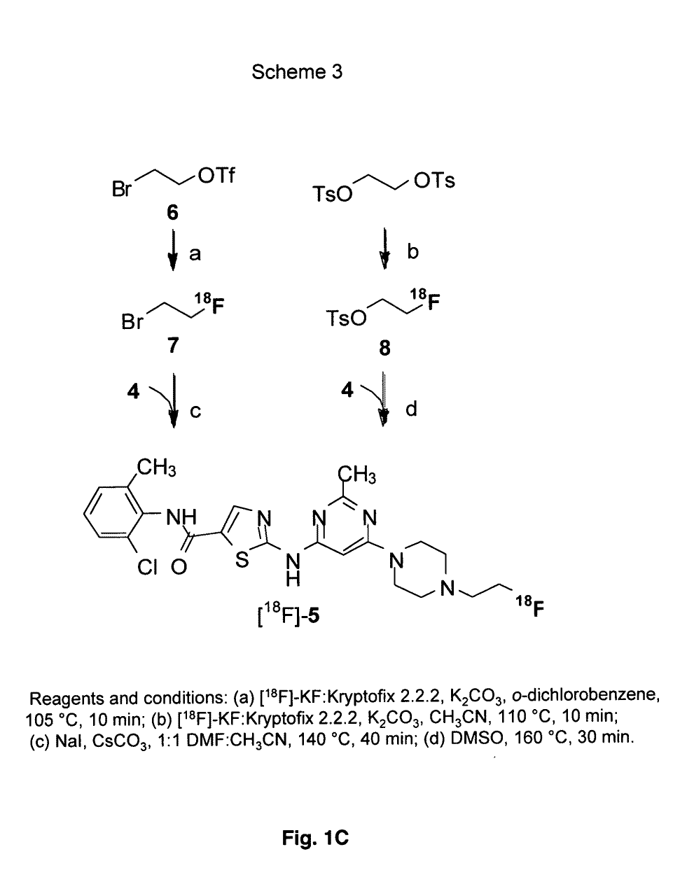

Dasatinib 1 was synthesized according to the procedure of Lombardo, et al (15). As shown in FIG. 1C (Scheme 1) dasatinib (free base) 1 (21 mg, 0.043 mmol) and triethylamine (12 μL, 0.086 mmol) and 1 mL of anhydrous DMF were added to a 5 mL screw-top vial under argon with magnetic stirring. The vial was cooled in an ice bath for 5 min. Methanesulfonyl chloride (5 μL, 0.065 mmol) was added and the mixture stirred 15 min, allowed to warm to ambient temperature and stirred 12 hours. The reaction mixture was partitioned between 20 mL of CH₂Cl₂ and 20 mL water. The organic layer was washed with water (2_{—}10 mL) and brine (10 mL), dried over Na₂SO₄, filtered and concentrated. The residue was resuspended in a small amount of CH₂Cl₂ and purification by gradient flash chromatography (SiO₂, 0% to 10% MeOH/CH₂Cl₂) yielded 12 mg (55%) of compound 2: ¹H NMR (DMSO-d₆) δ 11.46 (s, 1H), 9.86 (s, 1H), 8.21 (s, 1H), 7.38 (dd, 1H, J=7.6, 1.3 Hz), 7.29-7.22 (m, 2H), 6.04 (s, 1H), 3.71 (t, 2H, J=6.7 Hz), 3.51 (br s, 4H), 2.68 (t, 2H, J=6.6 Hz), 2.52 (br s, 4H) 2.40 (s, 3H), 2.23 (s, 3H); MS-ESI m/z 506.1 [M+H]⁺; HPLC t_{R}=12.0 min (Phenomenex Gemini C18 250×4.6 mm, gradient 70% A: 20 mM pH 4.1 KH₂PO₄/30% B: CH₃CN to 20% A/80% B, 1 mL/min, λ=254 nm). The fluoroethyl moiety is produced as described (49) to yield N-(2-Chloro-6-methylphenyl)-2-(6-(4-(2-fluoroethyl)piperazin-1-yl)-2-methylpyrimidin-4-ylamino)thiazole-5-carboxamide, 5. Radiofluorination of 5 is performed as described herein.

### Chemical Scheme 2

### N-(2-Chloro-6-methylphenyl)-2-(2-methyl-6-(piperazin-1-yl)pyrimidin-4-ylamino)thiazole-5-carboxamide, 4

2-(6-Chloro-2-methylpyrimidin-4-ylamino)-N-(2-chloro-6-methyl phenyl)thiazole-5-carboxamide, 3 (15) (1.00 g, 2.54 mmol), piperazine (2.19 g, 25.4 mmol) and N,N-diisopropylethylamine (0.84 mL, 5.07 mmol) were dissolved in 30 mL of dry 1,4-dioxane and refluxed overnight. The solvent was stripped and the residue was triturated several times with DI water/MeOH, MeOH/ether and ether. The white solid was dried under high vacuum to give precursor 4 (0.88 g, 78%). ¹H NMR (DMSO-d₆) δ 9.85 (s, 1H), 8.20 (s, 1H), 7.39 (dd, 1H, J=7.5, 1.5 Hz), 7.29-7.22 (m, 2H), 6.01 (s, 1H), 3.43 (m, 4H), 2.73 (m, 4H), 2.39 (s, 3H), 2.23 (s, 3H); ¹³C NMR (DMSO-d₆) δ 165.1, 162.6, 162.5, 159.9, 156.9, 140.8, 138.8, 133.5, 132.4, 129.0, 128.1, 127.0, 125.6, 82.4, 45.3 (2), 44.8 (2), 25.6, 18.3; FTIR (ATR) νₘₐₓ 3190, 2950, 1619, 1571, 1506, 1410, 1294, 1205, 1185, 769; MS-ESI m/z 445 [M+H]⁺; HRMS (FAB+) calc'd for C₂₀H₂₂ClN₇OS: 443.1295, found: 443.1303; HPLC t_{R}=2.6 min (Phenomenex Gemini C18 250×4.6 mm, 50% 20 mM pH 4.1 KH₂PO₄/50% CH₃CN, 1 mL/min, λ=254 nm).

### N-(2-Chloro-6-methylphenyl)-2-(6-(4-(2-fluoroethyl)piperazin-1-yl)-2-methylpyrimidin-4-ylamino)thiazole-5-carboxamide, 5

Piperazine 4 (50 mg, 0.11 mmol), 1-bromo-2-fluoroethane (21 μL, 0.27 mmol), K₂CO₃ (78 mg, 0.56 mmol), NaI (2 mg, 0.01 mmol) and 5 mL of CH₃CN were added to a 10 mL screw-top tube under argon. The vial was sealed and stirred 2 h at 60° C. Another 21 μL (0.27 mmol) of 1-bromo-2-fluoroethane (42) was added and the mixture stirred another 2 h. The reaction mixture was partitioned between 30 mL of EtOAc and 30 mL water. The organic layer was washed with water and brine, dried over MgSO₄ and concentrated to find a yellow oil. Purification by gradient flash chromatography (SiO₂, 0% to 10% 7N NH₃ in MeOH/CH₂Cl₂) yielded 46 mg (84%) of compound 5, a white powder: ¹H NMR (DMSO-d₆) δ 11.44 (s, 1H), 9.85 (s, 1H), 8.20 (s, 1H), 7.39 (d, 1H, J=7.0 Hz), 7.27-7.24 (m, 2H), 6.04 (s, 1H), 4.62-4.48 (dt, 4H, J=47.8, 4.8 Hz), 3.51 (m, 4H), 2.69-2.60 (m, 2H, dt, 4H, J=28.8, 4.8 Hz), 2.68 (m, 4H) 2.39 (s, 3H), 2.22 (s, 3H); ¹³C NMR (DMSO-d₆) δ 165.1, 162.5, 162.4, 159.9, 156.9, 140.8, 138.8, 133.5, 132.4, 129.0, 128.1, 127.0, 125.7, 82.6, 81.7 (d, J=164 Hz), 57.5 (d, J=19 Hz), 52.4 (2), 43.5 (2), 25.5, 18.3; ¹⁹F NMR (DMSO-d₆) δ −217; FTIR (ATR) νₘₐₓ 3202, 2945, 1622, 1576, 1504, 1413, 1394, 1290, 1188, 768; MS-ESI m/z 490 [M+H]⁺; HRMS (FAB+) calc'd for C₂₂H₂₆ClFNγOS: 489.1514, found: 489.1519; HPLC t_{R}=5.6 min (Phenomenex Gemini C18 250×4.6 mm, 50% 20 mM pH 4.1 KH₂PO₄/50% CH₃CN, 1 mL/min, λ=254 nm).

### Chemical Synthetic Scheme 3

### 2-Bromoethyltriflate 6

As shown in FIG. 1C, the radiosynthesis of [¹⁸F]-5 was performed via two methods from 2-bromoethyltriflate, 6 (Method A) or ethylene glycol ditosylate (Method B). 2-Bromoethyl triflate 6 has been used to install a [¹⁸F]-fluoroethyl moiety on piperazines before and is easily obtained by triflation of 2-bromoethanol and triflic anhydride (33). Production runs of [¹⁸F]-5 from 2-bromoethyltriflate had an average specific activity of 2,560 mCi/μmol (n=11) in 125±5 min after end-of-bombardment. Compound 6 was produced as in Chi, et al., distilled, aliquated, sealed under argon and stored at −20° C. (33)¹H NMR (CDCl₃-d) δ 4.75 (t, 2H, J=6.4 Hz), 3.61 (t, 2H, J=6.4 Hz); ¹³C NMR (CDCl₃-d) δ 118.5 (q, J=320 Hz), 74.2, 26.1; ¹⁹F NMR (CDCl₃-d) δ −75.0.

Method A: The QMA cartridge containing cyclotron-produced [¹⁸F] fluoride ion was eluted with a solution containing 420 μL of H₂O and 120 μL of 0.25 M K₂CO₃ into a 10 mL Reacti-vial containing 15 mg of Kryptofix [2.2.2] (4,7,13,16,21,24-hexaoxa-1,10-diazabicyclo[8.8.8]hexacosane) in 1.0 mL CH₃CN. Water was removed azeotropically with CH₃CN (3×1.0 mL) at 100-105° C. The Reacti-vial was cooled to 0° C. and to the anhydrous [¹⁸F] KF/K₂CO₃ complexed with Kryptofix (with [¹⁸F]-KF:Kryptofix 2.2.2) was added a solution of 2-bromoethyltriflate, 6, (0.054 mmole) in o-dichlorobenzene (500 μL) and heated to 105° C. for 10 min. The [¹⁸F]-1-bromo-2-fluoroethane ([¹⁸F]-7) formed was distilled at 120° C. by bubbling a stream of argon (100 mL/min) into another Reacti-Vial maintained at −25° C., containing a solution of piperazine precursor 4 (6.5 mg, 14.6 μM), NaI (9.0 mg, 60 μM), and Cs₂CO₃ (5 mg, 15.3 μM) in 500 μL of 1:1 CH₃CN:DMF. The activity in the receiving vial was measured periodically to follow the distillation procedure (5 min).

The Reacti-Vial was fitted with a new, un-pierced septum to minimize loss of [¹⁸F]-7 at high temperature. The solution was heated to 120° C. for 40 min, cooled, diluted with 1.2 mL of 1:4 CH₃CN:50 mM pH 5.5 NaOAc and passed through a 13 mm syringe filter (0.25 μm). This solution was injected onto a C₁₈ semi-preparative HPLC column and eluted under gradient conditions; 80% A (50 mM pH 5.5 NaOAc):20% B (CH₃CN) to 20% A:80% B. [¹⁸F]-5 eluted at 15.3 min, which was well resolved from precursor 4 (t_{R}=13.4 min). For intravenous administration, the product-containing fraction was stripped of solvent by rotary evaporation, formulated in 5% BSA in saline to the proper dosage and sterile filtered. Alternatively, the product-containing fraction was stripped of solvent by rotary evaporation, formulated in normal saline (0.9% NaCl) alone, 5% BSA in saline or aqueous Captisol (10% to 70% w/v beta-cyclodextrin sulfobutyl ether in water) to the proper dosage and sterile filtered.

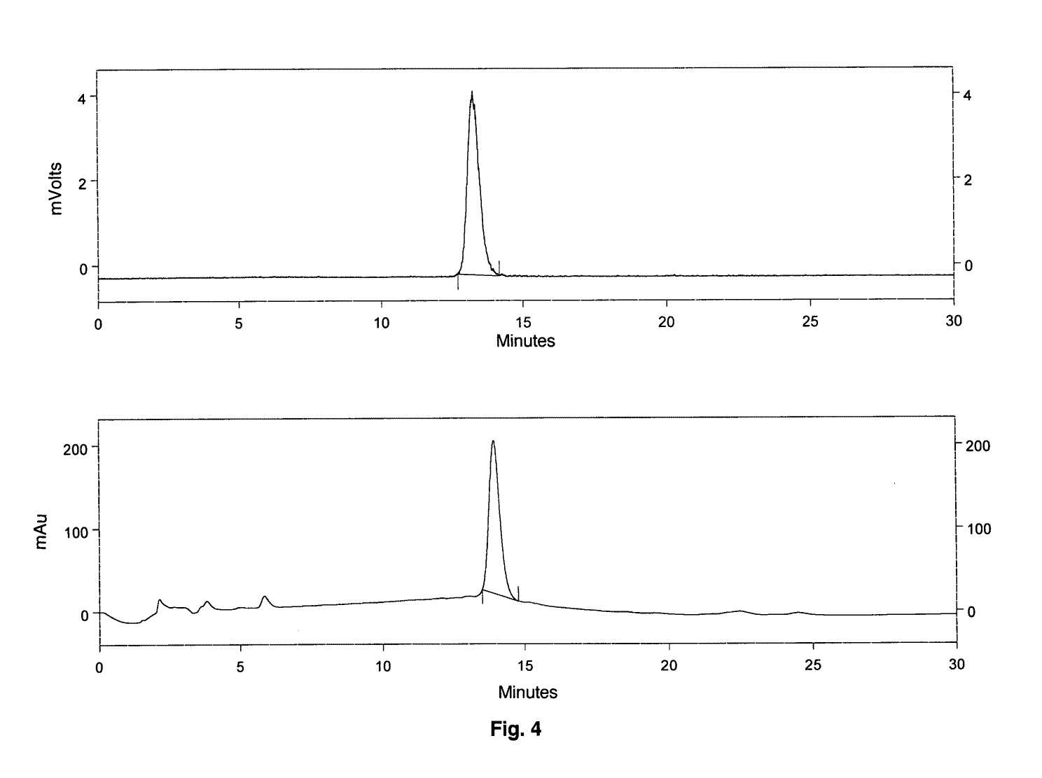

The radiochemical purity of the final formulation was confirmed using analytical HPLC. Co-elution with non-radioactive ¹⁸F reference compound 5 confirmed the identity of the radiotracer (FIG. 4). To measure radiochemical and chemical purity (>99%), [¹⁸F]-5 was reinjected from the semi-prep HPLC product peak on analytical HPLC (product t_{R}=13.2 min, isocratic 60% 50 mM pH 5.5 NaOAc:40% CH₃CN, 1.0 mL/min). Total time of radiosynthesis was 120±5 minutes from EOB. The decay-corrected radiochemical yields (n=3) were 25.1±5.8% from [¹⁸F]-1-bromo-2-fluoroethane and 6.6±2.3% overall from starting [18F]-fluoride. These conditions were optimized and it was found that [¹⁸F]-7 could be distilled rapidly prior to the alkylation of 4, which improved yields of [¹⁸F]-5 somewhat to 9.8±5.0%. The specific activity ranged from 108-7350 mCi/μmol (average 2560 mCi/μmol, n=11).

Method B: The QMA cartridge containing cyclotron-produced [¹⁸F] fluoride ion was eluted with a solution containing 420 μL of H₂O and 120 μL of 0.25 M K₂CO₃ (20 μmol) into a 5 mL Reacti-vial containing 10 mg (2.7 μmmol) of Kryptofix [2.2.2] in 0.5 mL CH₃CN. Water was removed azeotropically with CH₃CN (3×0.5 mL) at 105-110° C. To the anhydrous [¹⁸F] KF/K₂CO₃ complexed with Kryptofix was added a solution of ethylene glycol ditosylate (2.0 mg, 5.4 μmol) in CH₃CN (100 μL) and heated (sealed) in an oil bath at 110° C. for 10 min (35). The reaction mixture was cooled to room temp. and treated with a solution of 5.5 mg of piperazine precursor 4 in 100 μL of DMSO. The mixture was heated at 160° C. for 30 min, cooled, and passed through a C-18 Sep-Pak column activated previously with 8 mL of MeOH followed by 10 mL of DI water. The Sep-Pak was washed with water (2×6 mL) and [¹⁸F]-5 eluted with CH₃CN (1.2 mL). The Sep-Pak was washed with 0.8 mL of water and the combined [¹⁸F] solution purified and formulated as in method A. Total time of radiosynthesis was 120±10 minutes from EOB. The decay-corrected radiochemical yield was 23±5% (n=3) over two steps based on starting [¹⁸F]-fluoride. The specific activity was 3-6 mCi/μmol (n=3).

### [¹⁸F]-2-fluoroethyl tosylate 8

Compound 8 is generated in situ in a similar fashion from ethylene glycol ditosylate (35). The decay-corrected radiochemical yield of [¹⁸F]-5 from the tosylate, 8, was somewhat better over two steps (23%) but with much lower specific activity of 3-6 mCi/μmole (n=3). The total time of preparation (radiosynthesis and chromatography and formulation) ranged from 120 to 130 minutes (125±5 min). Compound 5 has a favorable log D_{(o/w)} of 2.1±0.6 and is highly protein bound in serum (98.5±1.0%) and 1% BSA (99.0±0.3%).

### Example 3

### Compound [¹⁸F]-5 kinase inhibition profile

Prior experience with pyridopyrimidinone Src/Abl inhibitors (34) and molecular docking studies into the Abl crystal structure predicted that the Dasatinib pharmacophore would share much of the same binding characteristics in which an arene sits deep within the catalytic pocket and the substitutents on N4 of the piperazine would protrude from a solvent accessible hole in the kinase catalytic domain (FIGS. 2A-2B). To determine whether compound 5 retains a kinase inhibition profile that is similar to Dasatinib, the inhibition of kinase activity was characterized. In vitro and cellular assays demonstrated that fluorinated analog 5 has inhibitory activity that closely parallels that of Dasatinib. Compound 5 inhibits Abl and Src kinase activity at roughly half the potency of Dasatinib in the assays (Table 1).

TABLE 1
Compound 5 has kinase and cellular inhibition
characteristics similar to Dasatinib.^{a}

| | Dasatinib | Compound 5 |
|---|---|---|
| | IC₅₀ (nM) | IC₅₀ (nM) |
| Abl protein | 4.2 ± 0.4 | 9.1 ± 0.8 |
| Src protein | 1.5 ± 1.1 | 3.5 ± 2.2 |
| K562 cells | 1.0 ± 0.2 | 1.1 ± 0.2 |
| R10 neg cells | 0.07 ± 0.02 | 0.10 ± 0.02 |
| (MO7e/p210^{bcr-abl}) | | |
| M07e cells^{b} | 1.2 ± 0.8 | 1.1 ± 0.3 |

^{a}Mean of three experiments in each.
^{b}Grown in 50 ng/mL SCF (Kit ligand)

Compound 5 is equipotent with Dasatinib in inhibiting proliferation of cells dependent on Bcr-Abl for growth. K562 growth was inhibited at an IC₅₀ of 1.1 nM and M07e/p210^{bcr-abl} cells at 0.10 nM. Also, Kit ligand dependent growth of the parental M07e line was inhibited with an IC₅₀ of 1.1 nM. This result correlates well with strong inhibition of Kit kinase as seen in the kinase panel.

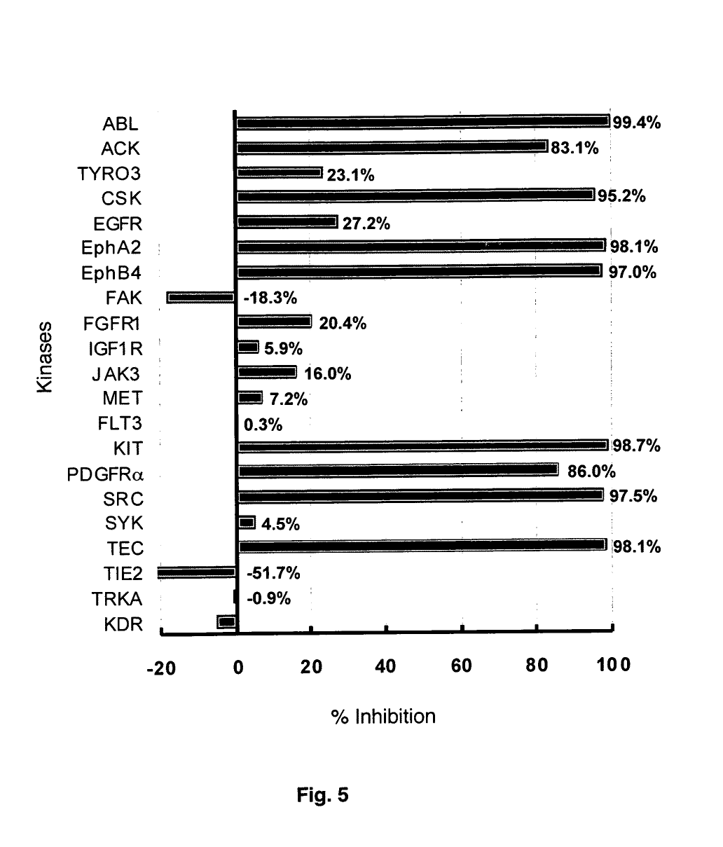

Kinase inhibition by 5 at 10 nM was examined in a panel of 21 kinases, which includes many relevant members for malignancies of interest (FIG. 5). As expected, the pattern of kinase binding data for Dasatinib (36) was very similar to the kinase inhibition profile of compound 5. Table 2 shows the inhibitory activity of Compound 5 and staurosporine against tyrosine kinases Negative values, particularly for TIE2 should be interpreted as an enhancement of substrate phosphorylation. Abl, Src and Kit are inhibited at >97% at 10 nM, which corresponds to IC₅₀'s of <2 nM. Furthermore, 5 potently inhibited Tec kinase and two representative ephrin receptor tyrosine kinases, EphA2 and EphB4. Recently, Tec and Btk kinases were found to be major targets of Dasatinib by chemical proteomics (37). While inhibiting the ephrin receptors may be a double-edged sword for therapeutics due to tumor-suppressor signaling (38), they are upregulated in a variety of cancers (39) and hold promise in molecular imaging (40). Table 3 shows EC50 and IC50 values of [¹⁸F]-5 and Dasatinib for various tyrosine kinases.

TABLE 2

| | % Inhibition of Kinase Activity | | | |
|---|---|---|---|---|
| | Compound | | Staurosporine | Dasatinib⁴ |
| Kinase | 5 (10 nM) | Staurosporine | conc. (nM) | K_{d} (nM) |
| ABL | 99.4 | 87.2 | (1000) | 0.50 |
| ACK | 83.1 | 96.4 | (30) | 6.0 |
| TYRO3 | 23.1 | 95.3 | (1000) | — |
| CSK | 95.2 | 89.3 | (300) | 1.0 |
| EGFR | 27.2 | 82.4 | (10000) | 100 |
| EphA2 | 98.1 | 92.6 | (10000) | 0.80 |
| EphB4 | 97.0 | 94.1 | (10000) | 0.30 |
| FAK | −18.3 | 81.7 | (100) | — |
| FGFR1 | 20.4 | 86.5 | (100) | 4000 |
| IGF1R | 5.9 | 96.9 | (3000) | >10000 |
| JAK3 | 16.0 | 95.3 | (3) | — |
| MET | 7.2 | 90.0 | (300) | — |
| FLT3 | 0.3 | 96.4 | (3) | 5000 |
| KIT | 98.7 | 96.7 | (10) | 0.60 |
| PDGFR_{—} | 86.0 | 88.4 | (3) | 0.40 |
| SRC | 97.5 | 89.8 | (3000) | 0.20 |
| SYK | 4.5 | 90.2 | (30) | 3000 |
| TEC | 98.1 | 92.5 | (300) | — |
| TIE2 | −51.7 | 90.9 | (300) | >10000 |
| TRKA | −0.9 | 94.0 | (3) | >10000 |
| KDR | −5.6 | 91.8 | (300) | 3000 |

TABLE 3

| | ^{[18]}F-5 | Dasatinib |
|---|---|---|
| Kinase | EC₅₀ (nM) | IC₅₀ (nM) |
| ABL | 0.60 | <1 |
| ACK | 20 | |
| CSK | 5.0 | |
| EGFR | 270 | 180 |
| EphA2 | 1.9 | |
| EphB4 | 3.1 | |
| FGFR1 | 390 | 880 |
| KIT | 1.3 | 5 |
| PDGFR_{—} | 16 | |
| SRC | 2.6 | 0.5 |
| TEC | 1.9 | |

Src/Abl is not selective and interacts with a number of kinases (36). A tumor overexpressing a particular kinase, such as Bcr/Abl or Src, can however selectively uptake a high-affinity probe in the presence of surrounding tissues that have negligible kinase expression. This selective uptake is possible with a related kinase-targeted radiotracer in Bcr-Abl overexpressing K562 cells (30).

### Compound [¹⁸F]-5 efficacy in vivo

In in vivo studies in mice, 5 has not shown toxicity at a dose of 0.1 mg/kg.

HPLC analysis of plasma samples revealed that [¹⁸F]-5 was metabolized significantly over a two-hour time course (see Table 2). Under these conditions three radioactive peaks were observed at 4.4, 15.1 and 16.9 minutes. Table 4 shows the metabolic profile of [¹⁸F]-5. The peak at 16.9 min corresponds to [¹⁸F]-5 whereas the other two are metabolites.

TABLE 4

| | Peak (% Radioactivity) | | |
|---|---|---|---|
| | | | 3 |
| | 1 | 2 | [¹⁸F]-5 |
| Time (min) | (4.4 min) | (15.1 min) | (16.9 min) |
| 30 | 58 ± 19 | 10 ± 9 | 31 ± 11 |
| 60 | 88 ± 4 | 3 ± 3 | 9 ± 3 |
| 120^{†} | 84 | 7 | 8 |

^{†}Only one trial was successful at this timepoint

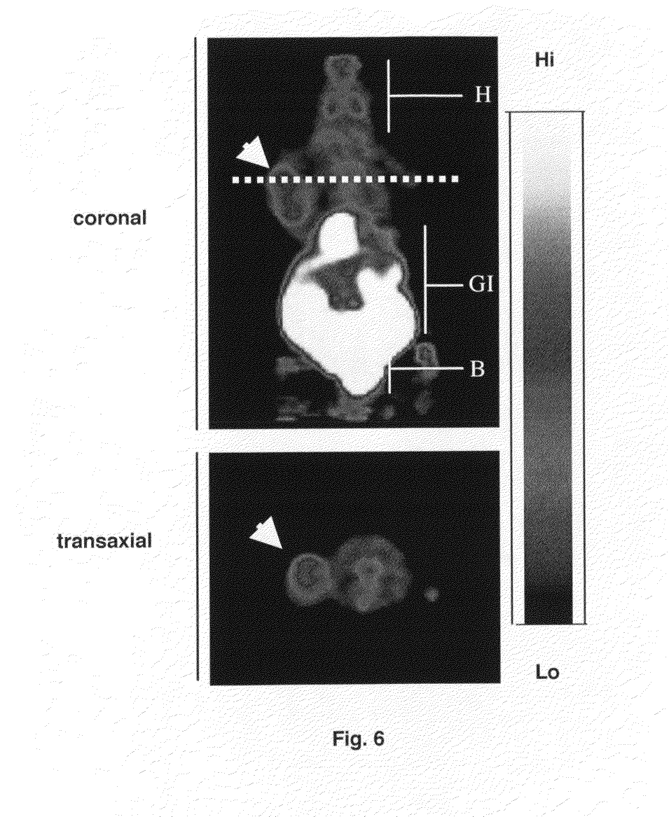

In vivo results have been obtained in athymic mice bearing subcutaneous K562 (CML) tumor xenografts in the right shoulder. [¹⁸F]-5 (14±1 MBq, 375±25 μCi) was injected through the tail vein and the subjects were imaged in a microPET scanner. FIG. 6 shows the microPET scan of one representative mouse 60 minutes after injection; the exposure time was 15 minutes. Tracer activity was evident within the tumor xenograft (white arrow) and was determined to be 1.1% of the injected dose by ROI (region of interest) analysis. The coronal image shows [¹⁸F]-5 activity in the tumor, blood pool activity in the head (H), physiologic excretion into the liver and gastrointestinal (GI) tract as well as into the kidneys and bladder (B). The transaxial image was taken at the level of the known palpable tumor as shown in the coronal section (broken line). The intensity of the radiotracer activity is color-graded as depicted by the colored scale. There was no significant uptake observed in bone, suggesting that [¹⁸F]-5 did not undergo rapid metabolic defluorination. Instead, the compound appears to be cleared via the hepatobiliary route predominantly. This result correlates with the distribution of Dasatinib in mice.

### Saturation of Available Target-Binding Sites in Tumors by Dasatinib

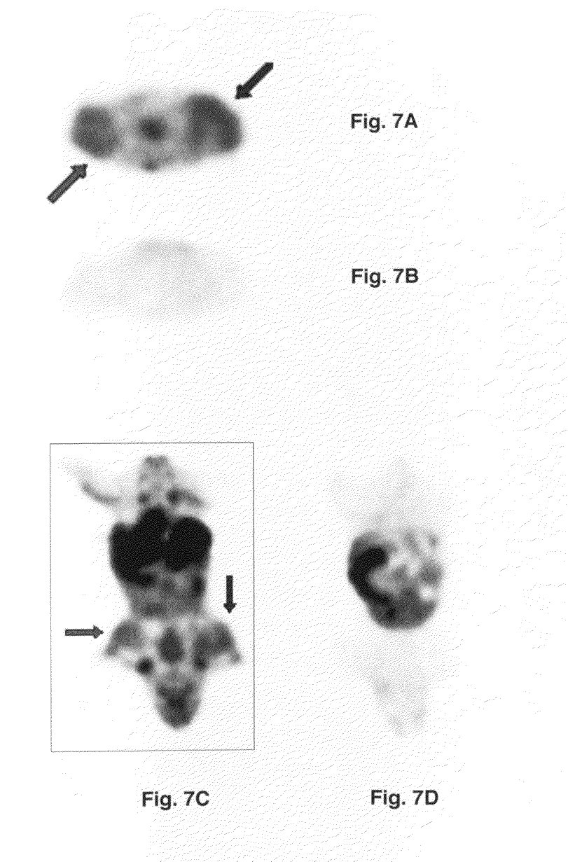

In vivo animal data demonstrates that therapeutic dosages of Dasatinib can completely saturate (or occupy) all available target-binding sites, in tumors. To determine if [¹⁸F]-5 uptake is competitively inhibited in vivo, a SCID mouse was injected with H1975 and H1975-DR lung cancer cells on its right and left shoulders, respectively (FIGS. 7A-7B). Transaxial and coronal images (FIGS. 7A, 7C) illustrate that tumor uptake is evident bilaterally. Unlabeled Dasatinib was administered (30 mg/kg IP) to the mouse. Transaxial (FIG. 7B) and coronal (FIG. 7D) images illustrate competitive inhibition of tracer uptake in the tumors and organs, e.g., skeleton, thereby confirming specificity of tracer binding.

Biodistribution of [¹⁸F]-5 solubilized with Captisol®

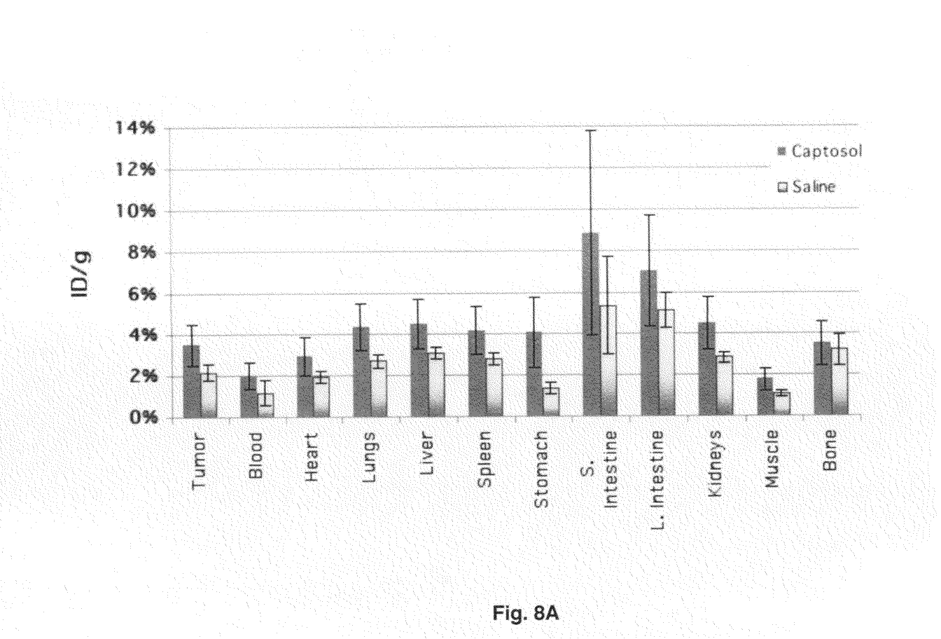

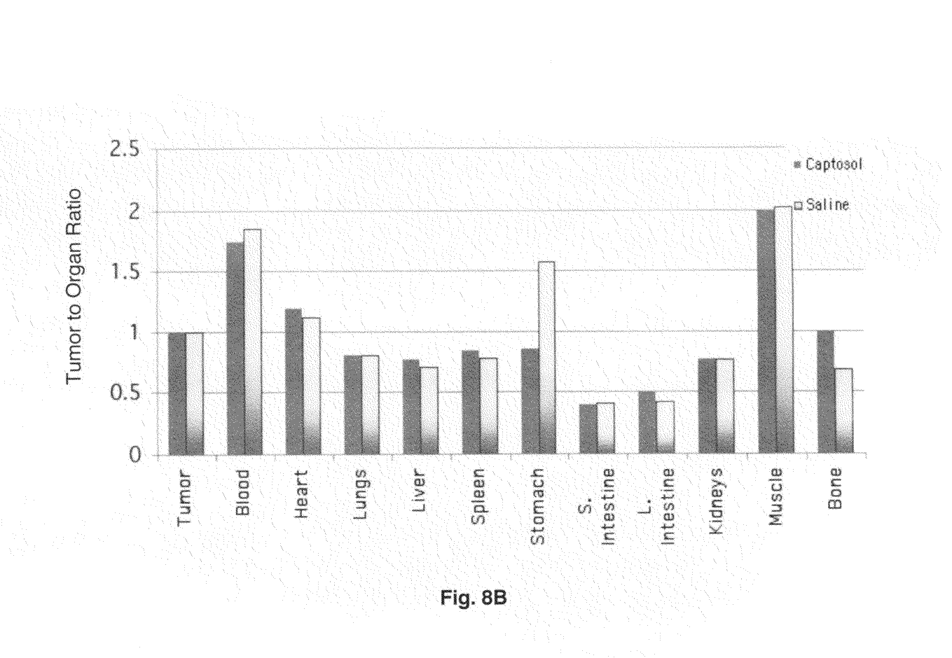

CWR22Rv1 prostate-tumor-bearing mice (n=5/group) were injected with compound [¹⁸F]-5 solubilized in Captisol® (CyDex) or with compound [¹⁸F]-5 in saline as control as described in Example 2. Average radioactivity was about 191 μCi. At the 60 min time point, the distribution of the radiotracer was nearly identical in the two groups with the net uptake slightly higher in the Captisol® group (FIG. 8A). Also, the tumor-to-organ ratios in the Captisol® and saline groups were very similar (FIG. 8B).

In addition it was observed that with the Captisol® formulation the residual syringe radioactivity was reduced to nearly undetectable levels during injection into the tumor-bearing mice. Thus, 99-100% of the measured dose of radiotracer was delivered to the mice compared to 86-95% transfer efficiency in the saline group. Thus the Captisol® formulation improves solubility of the hydrophobic radiotracer.

### Example 4

### Determination of Maximum Tumor Dose or Saturation

The cancer patient is injected with [¹⁸F]-Dasatinib, by intravenous bolus; and, after 1-2 hours, the patient lies upon a scanner bed for 20-30 minutes of imaging of the body in the PET camera. If the indication for [¹⁸F]-Dasatinib PET is to demonstrate tumor avidity for Dasatinib, a single pretreatment [¹⁸F]-Dasatinib PET would suffice. This indication is analogous to the use of ¹¹¹In-pentetreotide to predict tumor response to octreotide therapy; radioiodide scintigraphy to predict tumor response to radioiodide therapy; 99 mTc-bisphosphonate scintigraphy to predict response to bone-seeking radiopharmaceutical therapy, e.g., radiostrontium and radiosamarium; ¹²³I-MIBG to predict response to ¹³¹I-MIBG therapy; and so forth.

If the clinician wants to know whether a prescribed dose will saturate tumor target-receptors, patients are imaged twice: first, before the patient ever has been treated with Dasatinib, the patient is administered [¹⁸F]-Dasatinib and analyzed by PET scan to establish tumor Dasatinib-avidity and, second, during Dasatinib therapy, in which decreases in the [¹⁸F]-Dasatinib concentrations found in tumor reflect tumor target-receptor occupancy, by non labeled Dasatinib, and complete loss of tumor [¹⁸F]-Dasatinib uptake indicates tumor saturation with the non radioactive Dasatinib, indicating that higher therapeutic doses are not required to realized full Dastinib therapeutic outcome. If tumor saturation is visualized, the clinician may prescribe a lower dose of oral Dasatinib, as a lower prescribed dose may allow maximal anti-tumor efficacy with less risk of toxicity. If tumor saturation is not visualized, the clinician may prescribe a higher dose of oral Dasatinib, anticipating improved tumor response.

The following references are cited herein.
1. Hicks R., Cancer Imaging 2005, 5, 51-57.
2. Shields, A., Mol. Imaging. Biol. 2006, 8, 141-150.
3. Hernandez, et al., Trends Cell Biol. 2004, 14, 36-44.
4. Wang, J. Y. Oncogene 2000, 19, 5643-5650.
5. Wong, et al., Annu. Rev. Immunol. 2004, 22, 247-306.
6. Chen, et al., Anticancer Drugs 2006, 17, 123-131.
7. Deininger, et al., Blood 2005, 105, 2640-2653.
8. Zimmermann, et al. Bioorg. Med. Chem. Lett. 1997, 7, 187-192.
9. Hornick, et al., Hum. Pathol. 2007, 38, 679-687.
10. Melo, et al., Cancer Lett. 2007, 249, 121-132.
11. Barnes and Melo, Cell Cycle 2006, 5, 2862-2866.
12. Jiang et al., Leukemia 2007, 21, 926-935.
13. Tauchi, et al., Int. J. Hematol. 2006, 83, 294-300.
14. Weisberg, et al., Nat. Rev. Cancer 2007, 7, 345-356.
15. Lombardo, et al., J. Med. Chem. 2004, 47, 6658-6661.
16. Shah, et al., Science 2004, 305, 399-401.
17. Abourbeh, et al., Nucl. Med. Biol. 2007, 34, 55-70.
18. Bonasera, et al., Nucl. Med. Biol. 2001, 28, 359-374.
19. Dissoki, et al., J. Labelled Comp. Radiopharm. 2006, 49, 533-543.
20. Johnstrom, et al., J. Labelled Comp. Radiopharm. 1998, 41, 623-629.
21. Lim, et al., J. Labelled Comp. Radiopharm. 2000, 43, 1183-1191.
22. Lim, et al., J. Nucl. Med. 1998, 39, 20p-21p.
23. Wang, et al., Bioorg. Med. Chem. Lett. 2006, 16, 4102-4106.
24. Wang, et al., Bioorg. Med. Chem. Lett. 2005, 15, 4380-4384.
25. Larson, et al., J. Surg. Oncol. 1971, 3, 685-697.
26. Shreeve, et al., Exp. Hematol. 2007, 35, 173-179.
27. Agool, et al., J. Nucl. Med. 2006, 47, 1592-1598.
28. Ackermann, et al., J. Labelled Comp. Radiopharm. 2002, 45, 157-165.
29. Ackermann, et al., Nucl. Med. Biol. 2005, 32, 323-328.
30. Veach, et al., Nucl. Med. Biol. 2005, 32, 313-321.
31. Kil, et al., Nucl. Med. Biol. 2007, 34, 153-163.
32. Tokarski, et al., Cancer Res. 2006, 66, 5790-5797.
33. Chi, et al., J. Org. Chem. 1987, 52, 658-664.
34. Nagar, et al., Cell 2003, 112, 859-871.
35. Block, et al., J. Labelled Comp. Radiopharm. 1987, 24, 1029-1042.
36. Carter, et al., P. Natl. Acad. Sci. USA 2005, 102, 11011-11016.
37. Hantschel, et al., P. Natl. Acad. Sci. USA 2007, 104, 13283-13288.
38. Wang, J. Y., Nat. Cell Biol. 2006, 8, 785-786.
39. Noren, N. K., Cancer Res. 2007, 67, 3994-3997.
40. Cai, W., Eur. J. Nucl. Med. Mol. Imaging 2007, (published online Aug. 3, 2007). doi:10.1007/s00259-007-0503-5.
41. Baker, et al., J Biomed Sci 2006, 13, 499-507.
42. Nagar, et al., Cell 2003, 112, 859-871.
43. Tokarski, et al., Cancer Res. 2006, 66, 5790-5797.
44. Trentham, et al., Biochem. J. 1972, 126, 635-644.
45. Carter, et al., P. Natl. Acad. Sci. USA 2005, 102, 11011-11016.
46. Berman, et al., Leukemia Res. 2000, 24, 289-297.
47. Matsuguchi, et al., J. Biol. Chem. 1994, 269, 5016-5021.
48. Song et al, 2006. PMID: 16740687.
49. Ying et al., 2006, Molecular Imaging 5(3):368

Any patents or publications mentioned in this specification are indicative of the level of those skilled in the art to which the invention pertains. Further, these patents and publications are incorporated by reference herein to the same extent as if each individual publication was specifically and individually incorporated by reference.

One skilled in the art would appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. An [¹⁸F]-labeled compound for in vivo imaging of cells or tissue using positron emission tomography having a chemical structure: 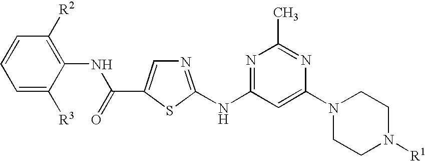
wherein R¹ is ¹⁸F, 1-piperazinyl-4-CH₂CH₂—¹⁸F or 1-piperazinyl-4-CH₂CH₂OCH₂CH₂—¹⁸F;
R² is CH₃ or ¹⁸F; and
R³ is Cl or ¹⁸F, such that only one of R¹, R² and R³ comprise an ¹⁸F.

2. The compound of claim 1, wherein R² is CH₃ and R³ is Cl.

3. The [¹⁸F]-labeled compound of claim 2, wherein R¹ is 1-piperazinyl-4-CH₂CH₂—¹⁸F, R² is CH₃ and R³ is Cl.

4. The [¹⁸F]-labeled compound of claim 1, wherein the compound is solubilized with a physiologically acceptable solubilizing agent or incorporated into a delivery vehicle.

5. The [¹⁸F]-labeled compound of claim 4, wherein the solubilizing agent is sulfobutyl beta-cyclodextrin, sulfated-beta-cyclodextrin or 2-hydroxypropyl-beta-cyclodextrin.

6. The [¹⁸F]-labeled compound of claim 4, wherein the delivery vehicle is a nanoparticle or a liposome.

7. A method for diagnosing a pathophysiological condition susceptible to treatment with a kinase inhibitor in a subject in need of such diagnosis, comprising the steps of:
administering a sufficient amount of the [¹⁸F]-labeled compound of claim 1 to the subject to provide an imageable concentration therewithin;
imaging the subject using positron emission tomography; and
determining whether the intensity of the label in any body area of the subject is increased in comparison with normal background, wherein an increase in intensity of the labeling indicates that the individual has a condition that is susceptible to being treated with the kinase inhibitor.

8. The method of claim 7, further comprising treating the pathophysiological condition with a pharmacologically effective dose of one or more of the kinase inhibitor.

9. The method of claim 8, further comprising:
synergistically treating the cancer by administering one or both of a chemotherapeutic agent or a radiotherapeutic agent.

10. The method of claim 9, further comprising monitoring the susceptibility of the pathophysiological condition to treatment with the kinase inhibitor to determine whether resistance or increased sensitivity to the treatment has developed.

11. The method of claim 10, wherein monitoring resistance or increased sensitivity to the treatment comprises the steps of:
a) administering another imageable amount of the compound to the subject;
b) imaging the subject using PET; and
c) comparing the intensity of the label in a body area associated with the pathophysiological condition to an immediately previously obtained label-intensity, wherein a decrease in intensity compared to the previous intensity indicates that the pathophysiological condition is more resistant to treatment with the kinase inhibitor(s) or wherein an increase in intensity compared to the previous intensity indicates that the pathophysiological condition is more sensitive to treatment.

12. The method of claim 11, further comprising repeating steps a) to c) to continue monitoring changes in resistance or sensitivity to treatment.

13. The method of claim 7, wherein the kinase inhibitor is dasatinib.

14. The method of claim 7, wherein the pathophysiological condition is a cancer or has an inflammatory component.

15. The compound of claim 7, wherein the pathophysiological condition is associated with a disregulated or an up-regulated kinase signaling pathway.

16. An in vivo method using positron emission tomography for imaging cells or tissue having a kinase activity associated with a pathophysiological condition in a subject, comprising the steps of:
administering to the subject a sufficient amount of the [¹⁸F]-labeled compound of claim 1 to provide an imageable concentration of the compound in the cells or tissue; and
detecting emissions from the [¹⁸F] label comprising the compound, thereby forming an image of the cells or tissue.

17. The method of claim 16, wherein the cells or tissue comprise a tumor.

18. The compound of claim 16, wherein the pathophysiological condition is a cancer or comprises an inflammatory component.

19. The compound of claim 16, wherein the pathophysiological condition is associated with a disregulated or an up-regulated kinase signaling pathway.

20. The compound of claim 16, wherein the kinase is a tyrosine kinase.

21. The compound of claim 20, wherein the tyrosine kinase is Abl, Ack, Csk, EphA2, EphB4, Kit, PDGFR-alpha, Src or Tec.

22. A method for maximizing tumor response to a kinase inhibitor with minimal toxicity therefrom in a subject having a cancer, comprising the steps of:
administering to the subject an imageable amount of the [¹⁸F]-labeled compound of claim 1;
imaging the subject a first time using positron emission tomography (PET);
administering to the subject a dose of the kinase inhibitor;
administering to the subject an imageable amount of the [¹⁸F]-labeled compound;
imaging the subject a second time using positron emission tomography (PET);
comparing the imaged tumor uptake of the [¹⁸F]-label in the second PET scan with the imaged tumor uptake of the [¹⁸F]-label in the first PET scan, wherein a disappearance of [¹⁸F]-label label intensity for any one tumor in the subject in the second scan compared to the intensity in the first scan indicates that the tumor is being treated at a sufficient therapeutic concentration of the kinase inhibitor and wherein if the intensity of the label remains the same or decreases, but does not disappear, the tumor requires an increase in the therapeutic dose of the kinase inhibitor, thereby maximizing tumor response.

23. The method of claim 22, further comprising designing a therapeutic regimen to treat the cancer with minimal toxicity to the subject based on the saturation dose of the kinase inhibitor.

24. The compound of claim 22, wherein the kinase is a tyrosine kinase.

25. The method of claim 24, wherein the kinase is Abl, Ack, Csk, EphA2, EphB4, Kit, PDGFR-alpha, Src or Tec.

26. A [¹⁸F] labeled compound having the chemical structure:

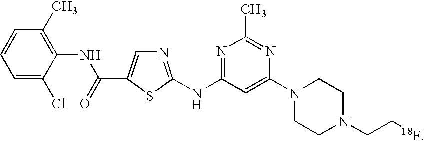

27. A composition comprising the [¹⁶F]-labeled compound of claim 26 and a physiologically acceptable excipient or delivery vehicle incorporating the compound.

28. The composition of claim 27, wherein the excipient is a sulfobutyl beta-cyclodextrin, sulfated-beta-cyclodextrin or 2-hydroxypropyl-beta-cyclodextrin.

29. The composition of claim 27, wherein the delivery vehicle is a nanoparticle or a liposome.

30. A solubilized radiotracer formulation, comprising the [¹⁸F] labeled compound of claim 26 and a physiologically acceptable solubilizing agent.

31. The solubilized radiotracer formulation of claim 3, wherein the solubilizing agent is a sulfobutyl beta-cyclodextrin, sulfated-beta-cyclodextrin or 2-hydroxypropyl-beta-cyclodextrin.
